(19) United States
(12) Patent Application Publication
(10) Pub. No.: US 2010/0010504 A1
(43) Pub. Date: Jan. 14, 2010

(54) SYSTEMS, DEVICES, AND METHODS FOR SURGERY ON A HOLLOW ANATOMICALLY SUSPENDED ORGAN

(75) Inventors: Nabil Simaan, New York, NY (US); Wei Wei, New York, NY (US); Roger Goldman, New York, NY (US); Howard Fine, New York, NY (US); Stanley Chang, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, NEW YORK (NY)

(21) Appl. No.: 12/441,566

(22) PCT Filed: Sep. 19, 2007
(86) PCT No.: PCT/US07/20281

§ 371 Date: Sep. 21, 2009

Related U.S. Application Data
(60) Provisional application No. 60/845,688, filed on Sep. 19, 2006.
(60) Provisional application No. 60/920,848, filed on Mar. 30, 2007.

(51) Int. Cl. A61B 19/00

(52) U.S. Cl. 606/130

(57) ABSTRACT

Systems, devices, and methods for surgery on a hollow anatomically suspended organ are described herein. In some embodiments a tele-robotic microsurgical system for eye surgery include: a tele-robotic master and a slave hybrid-robot; wherein the tele-robotic master has at least two master slave interfaces controlled by a medical professional; wherein the slave hybrid-robot has at least two robotic arms attached to a frame releasably attached to a patient's head; wherein the at least two robotic arms each have a parallel robot and a serial robot; and wherein the serial robot includes a tube housing a cannula.

### CROSS-REFERENCE TO RELATED APPLICATIONS

This application claims the benefit of U.S. Provisional Patent Applications No. 60/845,688, filed on Sep. 19, 2006 and 60/920,848, filed on Mar. 30, 2007, which are hereby incorporated by reference herein in their entireties.

### TECHNOLOGY AREA

Systems, devices, and methods for surgery on a hollow anatomically suspended organ are provided.

### BACKGROUND

Minimally invasive surgery on hollow anatomical suspended organs (e.g., ophthalmic microsurgery) presents medical professionals with unique challenges. Focusing on ophthalmic microsurgery, these challenges stem from the fact that the eye is a hollow and movable organ requiring very accurate and delicate surgical tasks to be performed inside it. During ophthalmic surgery, medical professionals use a microscope to visualize the retina by looking through the dilated iris. The tools currently used by medical professionals lack intraocular dexterity and are constrained to minimal degrees of freedom. Hence, it can be very difficult to perform complex ophthalmic surgery. Further, medical professionals can also be required to rotate the eye under the microscope in order to allow access to and visualization of the peripheral regions of the eye while manipulating multiple tools with very high precision.

The challenges of microsurgery include lack of intraocular dexterity of the tools, limited force feedback, and lack of depth perception when visualizing using the microscope. Microsurgery also demands a level of accuracy and bimanual dexterity not common to other surgical fields (e.g. positioning accuracy of 5-10 microns can be required). These difficult and precise bimanual tasks demonstrate the potential benefits and need for robotic assistance.

### SUMMARY

In some embodiments, a tele-robotic microsurgical system for eye surgery has: a tele-robotic master and a slave hybrid-robot; wherein the tele-robotic master has at least two master slave interfaces controlled by a medical professional; wherein the slave hybrid-robot has at least two robotic arms attached to a frame which is releasably attachable to a patient's head; and wherein the at least two robotic arms each have a parallel robot and a serial robot.

In some embodiments, a tele-robotic microsurgical system for eye surgery, has: a frame, a first robotic arm, a second robotic arm, and a tele-robotic master; wherein the frame is releasably attached to a patient's head; wherein the first robotic arm and second robotic arm each have a parallel robot and a serial robot; the tele-robotic master having a master slave interface controlled by a medical professional and the serial robot having a tube and a cannula.

In some embodiments, a tele-robotic microsurgical system for surgery on a hollow anatomically suspended organ, has: a tele-robotic master and a slave hybrid-robot; wherein the tele-robotic master has at least one master slave interface controlled by a medical professional; wherein the slave hybrid-robot has at least one robotic arm attached to a frame releasably attachable to a patient; and wherein the at least one robotic arm has a parallel robot and a serial robot.

In some embodiments, a slave-hybrid robot for surgery on a hollow anatomically suspended organ, has: a frame releasably attachable to a patient and at least one robotic arm releasably attached to the frame; wherein the at least one robotic arm has a parallel robot and a serial robot; wherein the serial robot has a tube for delivering a pre-bent NiTi cannula; wherein at least one of the tube and the pre-bent NiTi cannula is capable of rotating about its longitudinal axis; and wherein the pre-bent NiTi cannula is capable of bending when extended from the tube.

### DESCRIPTION OF DRAWINGS

The above and other objects and advantages of the disclosed subject matter will be apparent upon consideration of the following detailed description, taken in conjunction with accompanying drawings, in which like reference characters refer to like parts throughout, and in which:

FIG. 1A illustratively displays a method for using a tele-robotic microsurgery system in accordance with some embodiments of the disclosed subject matter;

FIG. 1B illustratively displays the general surgical setup for tele-robotic microsurgery on the eye in accordance with some embodiments of the disclosed subject matter;

FIG. 2 illustratively displays a slave hybrid-robot positioned over a patient's head in accordance with some embodiments of the disclosed subject matter;

FIG. 3 illustratively displays a tele-robotic microsurgical system for eye surgery including a tele-robotic master and a slave hybrid-robot in accordance with some embodiments of the disclosed subject matter;

FIG. 4A illustratively displays a slave hybrid-robot illustrating a serial robot and a parallel robot in accordance with some embodiments of the disclosed subject matter;

FIGS. 4B-4C illustratively display a serial connector included in a serial robot in accordance with some embodiments of the disclosed subject matter;

FIG. 5 illustratively displays a serial articulator included in a serial robot in accordance with some embodiments of the disclosed subject matter;

FIGS. 6A-6B illustratively display a tube for delivering a cannula in accordance with some embodiments of the disclosed subject matter;

FIG. 7 illustratively displays a slave hybrid-robot illustrating the legs of a parallel robot in accordance with some embodiments of the disclosed subject matter;

FIGS. 8-9 illustratively display an eye and an i^{th} slave hybrid-robot in accordance with some embodiments of the disclosed subject matter; and

FIGS. 10A-10B illustratively display an organ and an i^{th} slave hybrid-robot in accordance with some embodiments of the disclosed subject matter.

### DETAILED DESCRIPTION

In accordance with the disclosed subject matter, systems, devices, and methods for surgery on a hollow anatomically suspended organ are disclosed.

In some embodiments, a tele-robotic microsurgical system can have a slave hybrid robot having at least two robotic arms (each robotic arm having a serial robot attached to a parallel robot) and a tele-robotic master having at least two user controlled master slave interfaces (e.g., joysticks). Further, the serial robot for each robotic arm can have a tube housing a pre-bent NiTi cannula that is substantially straight when in the tube. Using each of the user controlled master slave interfaces, the user can control movement of the at least two robotic arms by controlling the parallel robot and serial robot for each robotic arm. That is, the user can control the combined motion of the serial robot and parallel robot for each arm by the master slave interfaces.

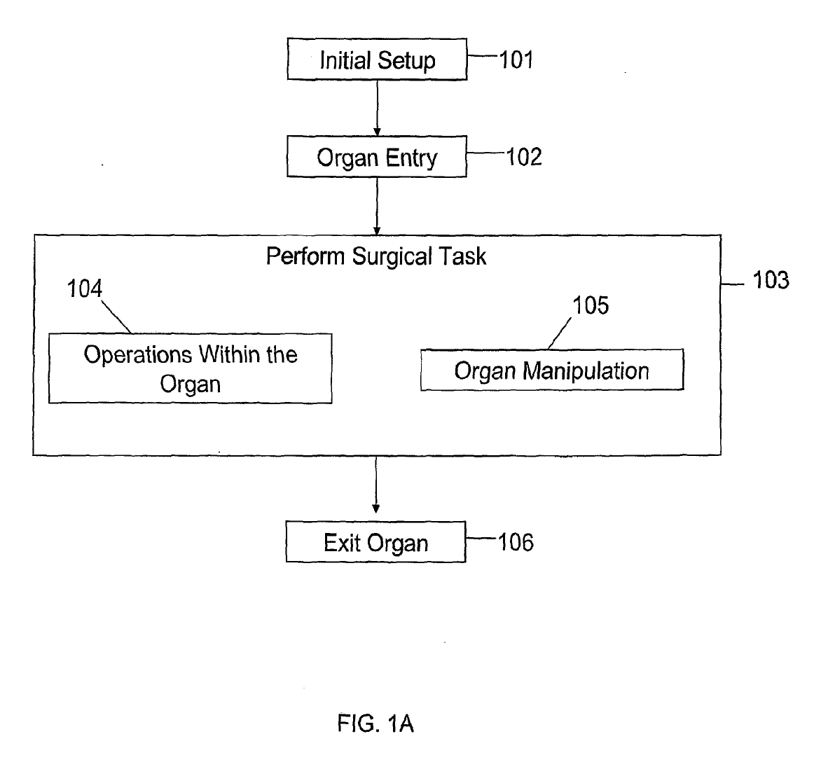

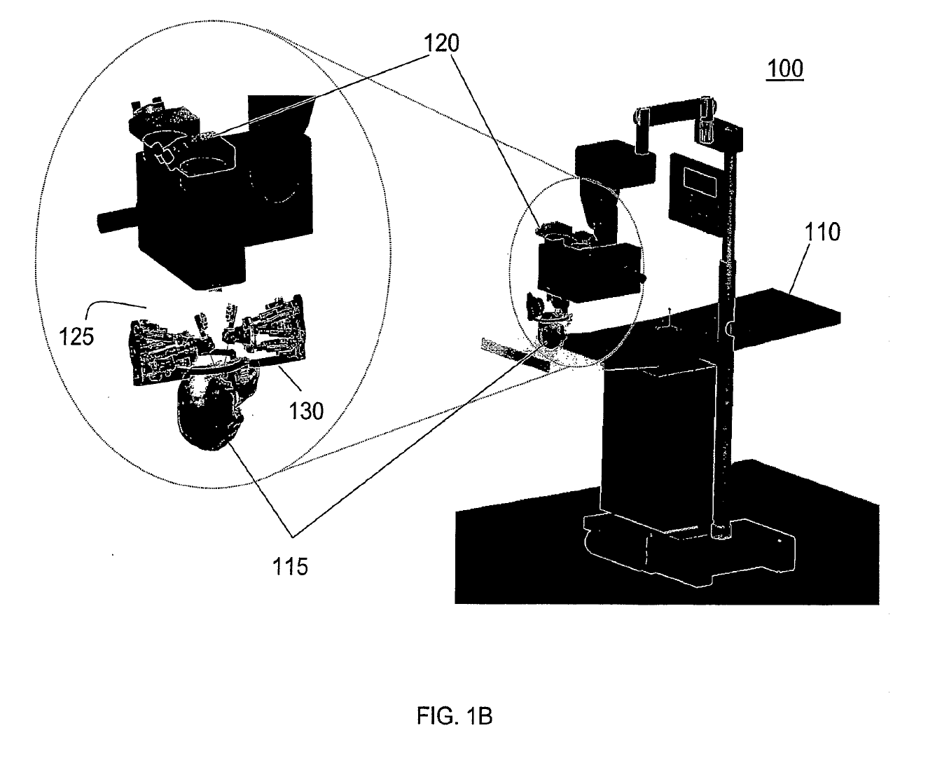

Referring to FIG. 1B, the general surgical setup for tele-robotic microsurgery on the eye is displayed. In some embodiments, a general surgical setup for eye surgery 100 includes a surgical bed 110, a surgical microscope 120, a slave hybrid-robot 125, and a tele-robotic master (not shown). The patient lies on surgical bed 110, with his head 115 positioned as shown. During eye surgery a patient located on surgical bed 110, has a frame 130 releasably attached to their head, and a slave hybrid-robot releasably attached to frame 130. Further, a medical professional can look into the patient's eye through surgical microscope 120 and can control drug delivery, aspiration, light delivery, and delivery of at least one of microgrippers, picks, and micro knives by the tele-robotic master which is in communication with slave hybrid-robot 125.

Referring to FIG. 1A a method for using a tele-robotic microsurgical system is illustratively displayed. For initial setup (101 in FIG. 1A), the slave-hybrid robot can be positioned over the organ (e.g., attached to a frame connected to the head of a patient). For example, a slave-hybrid robot having a first robotic arm (having a first parallel robot and first serial robot) and a second robotic arm (having a second parallel robot and a second serial robot) can have both arms in a position minimizing the amount of movement needed to enter the organ. For organ entry (102 in FIG. 1A), using a first user controlled master slave interface to control the first robotic arm, the user can insert a first tube, housing a first pre-bent NiTi cannula, into a patient's organ by moving the first parallel robot. Similarly, using a second user controlled master slave interface to control the second robotic arm, the user can insert a second tube into the patient's organ by moving the second parallel robot.

Inside the organ the user can perform surgical tasks (103 in FIG. 1A), such as organ manipulation (105 in FIG. 1A) and operations inside the organ (104 in FIG. 1A). Organ manipulation (105 in FIG. 1A) and operations inside the organ (104 in FIG. 1A) can occur in series (e.g., operations inside the organ then organ manipulation, organ manipulation then operations inside the organ, etc.) or in parallel (e.g., operations inside the organ and organ manipulation at substantially the same time).

For example, performing operations inside the organ (104 in FIG. 1A) and organ manipulation (105 in FIG. 1A) in series is described below. For performing operations inside the organ (104 in FIG. 1A), using the first user controlled master slave interface to control the first robotic arm, the user can control the first serial robot extending the first pre-bent NiTi cannula out of the first tube, the first pre-bent NiTi cannula bending as it exits the first tube. This bending represents one degree of freedom for the serial robot as described below. Further, using the first user controlled master slave interface to control the first robotic arm, the user can use the first serial robot to rotate at least one of the first pre-bent NiTi cannula and the first tube about their longitudinal axis (hence positioning the NiTi cannula inside the organ). This rotation about the longitudinal axis represents a second degree of freedom for the serial robot. Similarly, using the second user controlled master slave interface to control the second robotic arm, the user can use the second serial robot to move a second pre-bent NiTi cannula out of the second tube. The second pre-bent NiTi cannula bends as it exits the second tube. Again, similarly, the user can rotate at least one of the second pre-bent NiTi cannula and the second tube about their longitudinal axis. In some instances, delivering a second pre-bent NiTi cannula out of the tube is not necessary. For example, the second tube can be used for delivering a light into the organ. Further, for example, the pre-bent NiTi cannula can be delivered outside of the tube to provide a controlled delivery of light through an embedded optical fiber. Further still, for example, the pre-bent NiTi cannula can be delivered outside of the tube to provide a controlled delivery of an optical fiber bundle for controllable intra-ocular visualization for applications such as visualizing the distance between tools and the retina by providing a side view to the surgeon.

Further, for performing operations within the organ (104 in FIG. 1A), the user can utilize at least one of the first and second NiTi cannula and first and second tubes for drug delivery, aspiration, light delivery, and delivery of at least one of microgrippers, picks, and micro knives into the organ. The user can manipulate and position the organ (105 in FIG. 1A), with both tubes in the patient's organ,. For example, using both the first and second user controlled master slave interfaces, the user can move both parallel robots together (hence moving the tubes in the organ) and manipulate the organ. Further, after manipulating the organ the user can perform additional operations within the organ (104 in FIG. 1A).

For exiting the organ (106 in FIG. 1A), that is, to remove the surgical instruments from the organ, the user uses the first, user controlled master slave interface to control the first robotic arm. The user retracts the first pre-bent NiTi cannula into the first tube using the first serial robot. For instances where a second pre-bent NiTi cannula has been delivered, the user can similarly retract the second pre-bent NiTi cannula into the second tube using the serial robot. Using both the first and second user controlled master slave interfaces to control respectively the first and second robotic arms, the user can move both the first and second parallel robots to retract both the first and second tubes from the organ. In cases of emergency the serial robots can be removed from the eye by releasing a fast clamping mechanisms connecting them to the parallel robots and subsequently removing the frame with the two parallel robots.

It will be apparent that the disclosed subject matter can be used for surgery on any hollow anatomically suspended organs in the body. For example, the disclosed subject matter can be used on the eye, heart, liver, kidneys, bladder, or any other substantially hollow anatomically suspended organ deemed suitable. For ease in understanding the subject matter presented herein, the following description focuses on tele-robotic microsurgery on the eye.

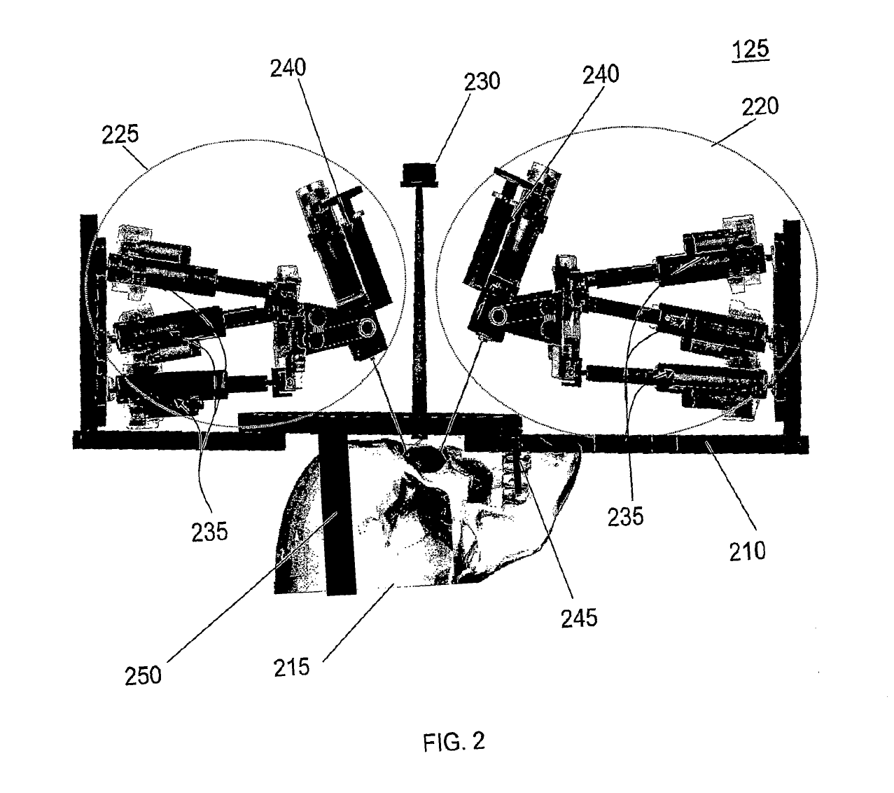

Referring to FIG. 2, a slave hybrid-robot 125 positioned over a patient's head is displayed. In some embodiments, the slave hybrid-robot 125 can be attached to a frame 210 which in turn is attached to a patient's head 215. Further, slave hybrid-robot 125 includes a first robotic arm 220 and a second robotic arm 225 that can be attached to frame 210 and can further include a microscope/viewcone 230. Still further, in some embodiments, first robotic arm 220 and second robotic arm 225 can include a parallel robot 235 (e.g., a Stewart platform, Stewart/Gough platform, delta robot, etc.) and a serial robot 240 (e.g., a robot consisting of a number of rigid links connected with joints). Some parts of the first and second robotic arms can be permanently attached to the frame while other parts can be releasably attached to the frame. Further, the serial robot can be releasably attached to the parallel robot. For example, for a robotic arm including a parallel and a serial robot, the parallel robot can be permanently attached to the frame and the serial robot can be releasably attached to the parallel robot. In some embodiments, the serial robot can be releasably attached to the parallel robot by, for example, lockable adjustable jaws.

In some embodiments, the slave hybrid-robot includes at least two robot arms releasably attached to the frame. For example, the robot arms can be attached to the frame by an adjustable lockable link, a friction fit, a clamp fit, a screw fit, or any other mechanical method and apparatus deemed suitable. Further, the robotic arms can be permanently attached to the frame. For example, the robotic arms can be attached by welding, adhesive, or any other mechanism deemed suitable.

In some embodiments, first robotic arm 220 and second robotic arm 225 can be adjusted into location at initial setup of the system (e.g., at the beginning of surgery). This can be done, for example, to align the robotic arms with the eye. Further, first robotic arm 220 and second robotic arm 225 can have a serial robot and a parallel robot where only one of the serial robot or parallel robot can be adjusted into location at initial setup of the system.

In some embodiments, frame 210 can be attached to the patient's head by a bite plate 245 (e.g., an item placed in the patient's mouth which the patient bites down on) and a surgical strap 250. Frame 210 can be designed to produce the least amount of trauma to a patient when attached. For example, frame 210 can be attached to a patient's head by a coronal strap (e.g., a strap placed around the patient's head) and a locking bite plate (e.g., a bite plate which can be locked onto the patient's mouth where the bite plate locks on the upper teeth ). Any mechanism for attaching the frame to the patient's head can be used. For example, the frame can be attached to the patient's head by a bite plate, surgical strap, or tension screw. Further, frame 210 can be screwed directly into the patient's skull.

Further, bite plate 245 can include air and suction access (not shown). For example, in an emergency, first robotic arm 220 and second robotic arm 225 can be released from the frame and the patient can receive air and suction through tubes (not shown) in the bite plate access.

Frame 210 can be made using a substantially monolithic material constructed in a substantially circular shape with a hollow center. Further, the shape of frame 210 can be designed to fit the curvature of the patient's face. For example, the frame 210 can be substantially round, oval, or any other shape deemed suitable. The frame material can be selected to be fully autoclaved. For example, the frame material can include a metal, a plastic, a blend, or any other material deemed suitable for an autoclave. Further still, frame 210 can include a material that is not selected to be fully autoclaved. That is, the frame can be for one time use.

In some embodiments, first robotic arm 220 and second robotic arm 225 include hybrid-robots. It will be understood that a hybrid-robot refers to any combination of more than one robot combined for use on each of the robotic arms. For example, in some embodiments, first robotic arm 220 and second robotic arm 225 include a six degree of freedom parallel robot (e.g., a Stewart platform, Stewart/Gough platform, delta robot, etc.) attached to a two degree of freedom serial robot (e.g., an intra-ocular dexterity robot) which when combined produce 16 degrees of freedom in the system. The hybrid-robots can include a parallel robot with any number of degrees of freedom. Further, the two degree of freedom serial robot (e.g., intra-ocular dexterity robot) can provide intra-ocular dexterity while the parallel robot can provide global high precision positioning of the eye and any surgical tool inside the eye. Still further, the hybrid-robots can include any combination of robots including a serial robot, parallel robot, snake robot, mechanatronic robot, or any other robot deemed suitable.

First robotic arm 220 and second robotic arm 225 can be substantially identical. For example, both first robotic arm 220 and second robotic arm 225 can include a parallel robot and a serial robot. Further, first robotic arm 220 and second robotic arm 225 can be substantially different. For example, first robotic arm 220 can include a first parallel robot attached to a second serial robot while second robotic arm 225 can include a first parallel robot attached to a second parallel robot.

In some embodiments, slave hybrid-robot 125 includes only two robotic arms. Using two robotic arms increases the bimanual dexterity of the user. For example, the two robotic arms can be controlled by a medical professional using two user controlled master slave interfaces (e.g., one controller in contact with each hand). Further, more than two robotic arms can be used in slave hybrid-robot 125. For example, four robotic arms can be used in slave hybrid-robot 125. Any suitable number of robotic arms can be used in slave hybrid-robot 125.

The robotic arms can be constructed to be reused in future operations. For example, first robotic arm 220 and second robotic arm 225 can be designed to be placed in an autoclave. Further, first robotic arm 220 and second robotic arm 225 can be designed for one time use. For example, first robotic arm 220 and second robotic arm 225 can be designed as throw away one time use products. Still further, parts of the robotic arms can be designed for one time use while other parts can be designed to be used in future operations. For example, first robotic arm 220 and second robotic arm 225 can include a disposable cannula, which can be used one time, and a reusable parallel robot.

In some embodiments, the slave hybrid-robot can be designed to use less than 24 Volts and 0.8 Amps for each electrical component. Using less than 24 Volts and 0.8 Amps can minimize safety concerns for the patient. Further, in some embodiments, both the parallel robot and serial robot allow sterile draping and the frame supporting the parallel and serial robot can be designed to be autoclaved.

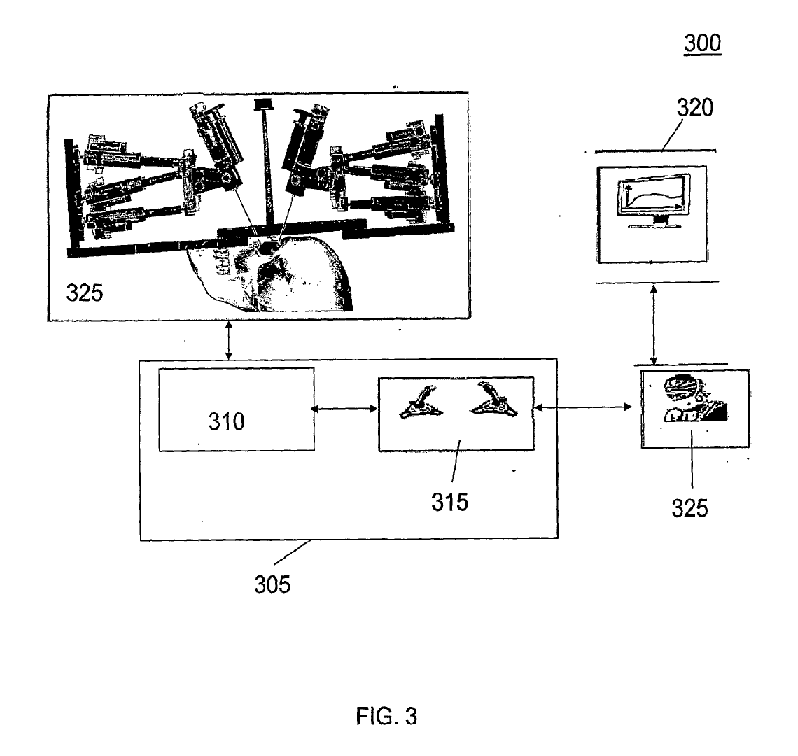

Referring to FIG. 3, in some embodiments, a tele-robotic microsurgical system for eye surgery 300 includes a tele-robotic master 305 and a slave hybrid-robot 325. In some embodiments, tele-robotic robotic master 305 includes a controller 310 and a user controlled master slave interface 315 (e.g., two force feedback joysticks). In some embodiments, controller 310 includes at least one of a dexterity optimizer, a force feedback system, and a tremor filtering system.

The force feedback system can include a display 320 for indicating to a medical professional 325 the amount of force exerted by the robotic arms (e.g., the force on the cannula in the eye). Further, the force feedback system can include providing resistance on user controlled master slave interface 315 as the medical professional increases force on the robotic arms. Further still, at least one of the robotic arms can include a force sensor and torque sensor to measure the amount of force or torque on the arms during surgery. For example, at least one of the robotic arms can include a 6-axis force sensor for force feedback. These sensors can be used to provide force feedback to the medical professional. Forces on the robotic arms can be measured to prevent injuring patients.

A tremor reducing system can be included in robotic master 305. For example, tremor reduction can be accomplished by filtering the tremor of the surgeon on the tele-robotic master side before delivering motion commands. For example, the motions of a master slave interface (e.g., joystick) can be filtered and delivered by the controller as set points for a PID (proportional, integral, and differential) controller of the slave hybrid-robot. In this example the two tilting angles of the master joystick can be correlated to axial translations in the x-and y directions. The direction of the master slave interface (e.g., joystick) can be correlated to the direction of movement of the slave in the x-y plane while the magnitudes of tilting of the master slave interface (e.g., joystick) can be correlated to the magnitude of the movement velocity of the robotic slave in x-y plane. In another embodiment the user can control the slave hybrid robot by directly applying forces to a tube (described below) included in the serial robot. Further, the serial robot can be connected to the parallel robot through a six-axis force and moment sensor that reads forces that the user applies and can deliver signals to the controller 310 that translates these commands to motion commands while filtering the tremor of the hand of the surgeon. Any suitable method for tremor reducing can be included in tele-robotic master 305. For example, any suitable cooperative manipulation method for tremor reducing can be used.

In some embodiments, a dexterity optimizer can include any mechanism for increasing the dexterity of the user. For example, the dexterity optimizer can utilize a preplanned path for entry into the eye. In some embodiments, the dexterity optimizer takes over the delivery of the tube into the eye by using the preplanned path.

The tele-robotic master and slave hybrid-robot can communicate over a high-speed dedicated Ethernet connection. Any communications mechanism between the tele-robotic master and slave hybrid-robot deemed suitable can be used. Further, the medical professional and the tele-robotic master can be in a substantially different location than the slave hybrid-robot and patient.

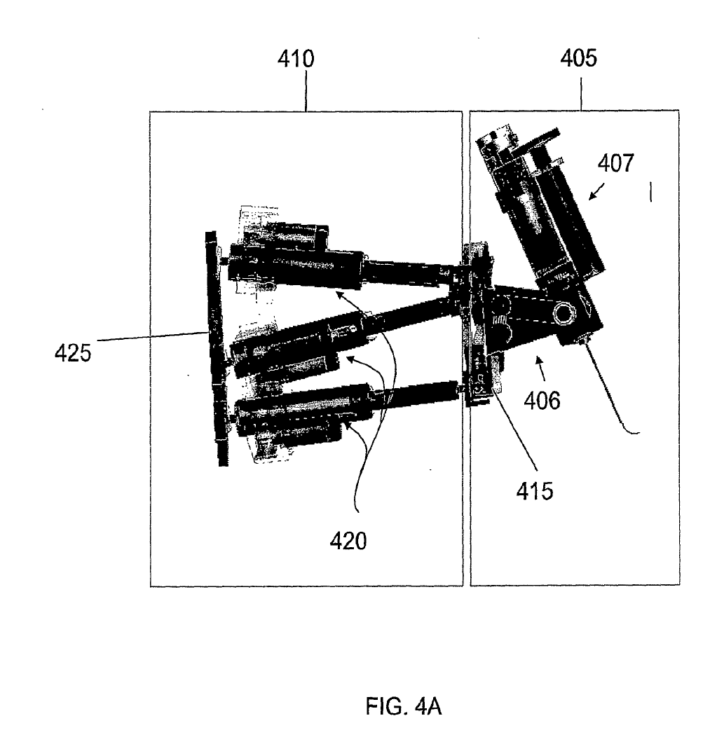

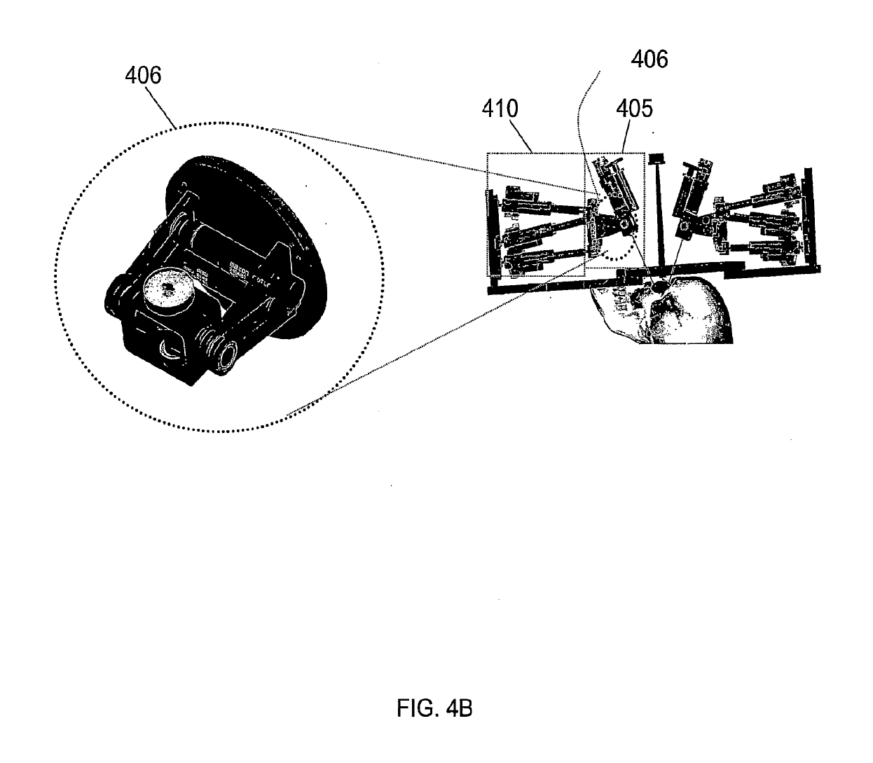

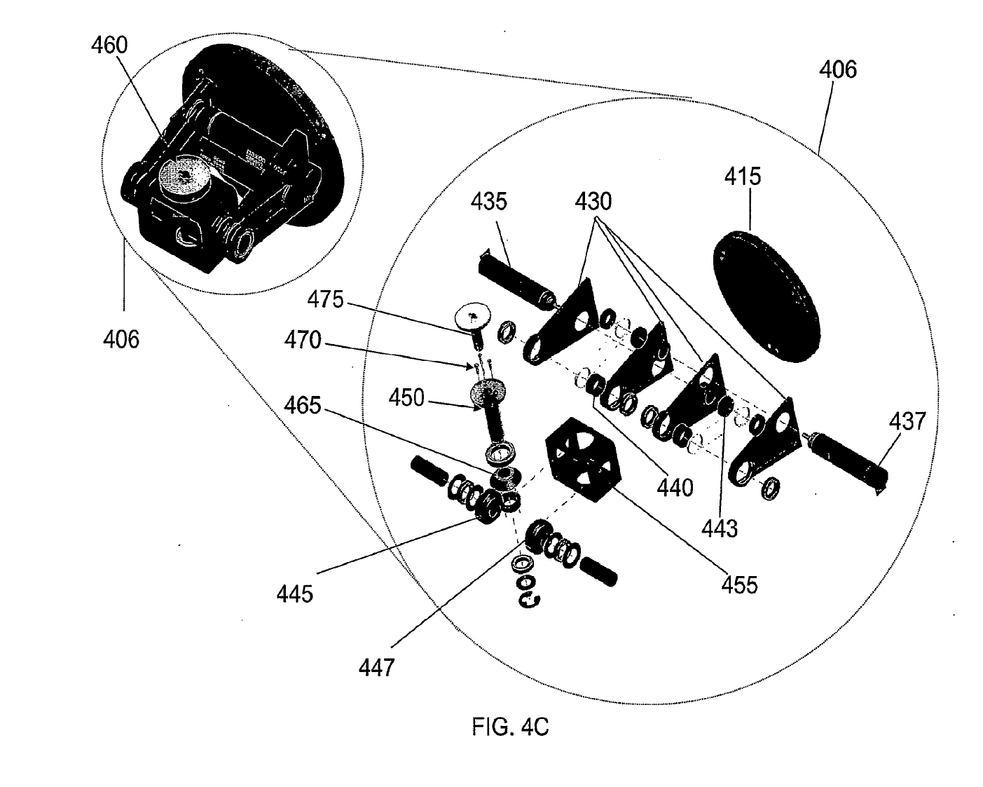

Referring to FIG. 4A, in some embodiments, the slave hybrid-robot can include a serial robot 405 and a parallel robot 410. Further, in some embodiments, serial robot 405 can include a serial connector 406 for connecting a platform 415 (e.g., the parallel robot's platform) and a serial articulator 407. Any mechanical connection can be used for connecting the parallel robot's platform and serial articulator 407. Platform 415 can be connected to legs 420 which are attached to base 425.

Referring to FIG. 4B, a serial robot 405 including serial connector 406 is illustratively displayed. The serial connector can be enlarged for a clearer view of the serial connector. Referring to FIG. 4C, an exploded view of serial connector 406 is displayed for a clearer view of a possible construction for serial connector 406. Any suitable construction for serial connector 406 can be used. For example, serial connector 406 can connect serial articulator 407 (FIG. 4A) with parallel robot 410 (FIG. 4A). Referring to FIG. 4C, platform 415 (e.g., the parallel robot moving platform) can support hollow arms 430 that can support a first electrical motor 435 and a second electric motor 437. First electric motor 435 and second electric motor 437 can actuate a first capstan 440 and a second capstan 443 via a first wire drive that actuate anti-backlash bevel gear 445 and a second wire drive actuate anti-backlash bevel gear 447 that can differentially actuate a third bevel gear 465 about its axis and tilt a supporting bracket 455. Differentially driving first electric motor 435 and second electric motor 437, the tilting of bracket 455 and the rotation of a fast clamp 460 about the axis of the cannula can be controlled.

Further referring to FIG. 4C, an exploded view of the fast clamp 460 is displayed for a clearer view of a possible construction for fast clamp 460. Fast clamp 460, included in serial connector 406, can be used to clamp instruments that are inserted through the fast clamp 460. Any suitable construction for fast clamp 460 can be used. For example, fast clamp 460 can include a collet housing 450, connecting screws 470, and a flexible collet 475. Connecting screws 470 can connect collet housing 450 to third bevel gear 450. Collet housing 450 can have a tapered bore such that when flexible collet 475 is screwed into a matching thread in the collet housing 450 a flexible tip (included in flexible collet 475) can be axially driven along the axis of the tapered bore, hence reducing the diameter of the flexible collet 475. This can be done, for example, to clamp instruments that are inserted through the fast clamp 460. Any other suitable mechanism for clamping instruments can be used.

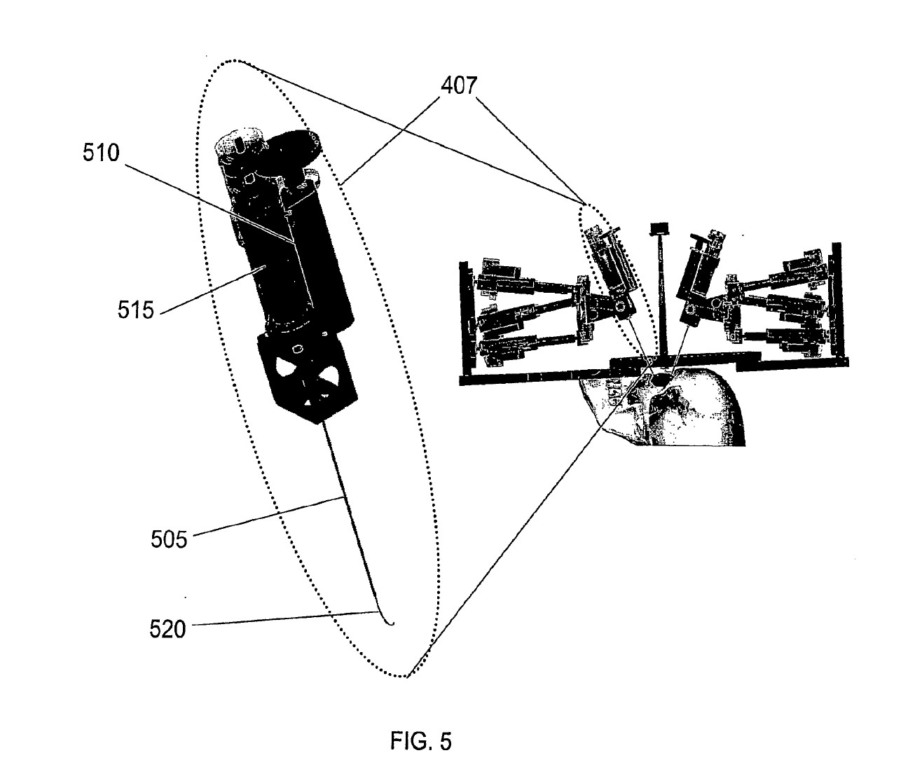

Referring to FIG. 5, in some embodiments, the serial robot includes a serial articulator 407 for delivering at least one of a tube 505 and a cannula 520 into the eye. In some embodiments, for example, serial robot articulator 407 includes a servo motor 510 and high precision ball screw 515 for controlling delivery of at least one of tube 505 and cannula 520. Servo motor 510, coupled to high-precision ball screw 515, can add a degree of freedom to the system that can be used for controlling the position of cannula 520 with respect to tube 505. For example, servo motor 510 can be coupled to a hollow lead screw (not shown) that when rotated drives a nut (not shown) axially. Further, for example, cannula 520 can be connected to the nut and move up/down as servo motor 510 rotates the lead screw (not shown). Any suitable mechanism for controlling the delivery of tube 505 and cannula 520 can be used. Further, in some embodiments, tube 505 houses cannula 520.

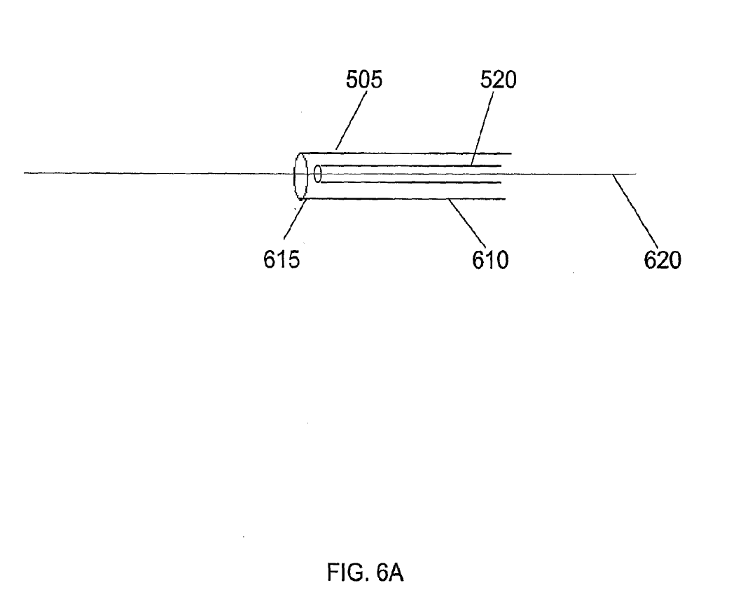

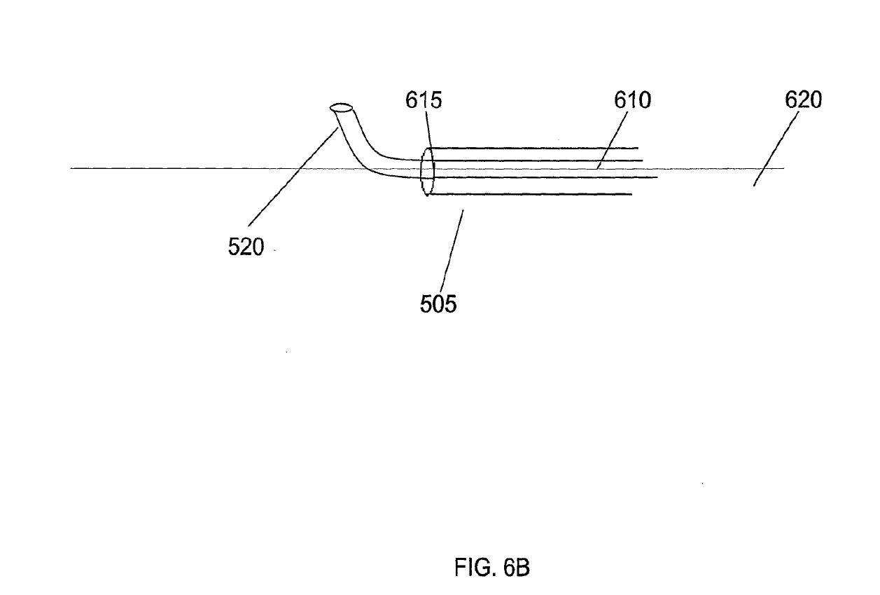

Referring to FIGS. 6A-6B, in some embodiments, cannula 520 can be delivered through tube 505 into the eye. FIG. 6A illustratively displays a cannula 520 in a straightened position while housed in tube 505. FIG. 6B illustratively displays cannula 520 in a bent position as cannula 520 has exited tube 505 (hence the cannula has assumed its pre-bent shape). The pre-bent shape of cannula 520 can be created by using any shape memory alloy (e.g., NiTi) and setting the shape so that the cannula assumes the bent position at a given temperature (e.g., body temperature, room temperature, etc.). Further, although cannula 520 is described as having a specific pre-bent shape, any shape deemed suitable can be used (e.g., s-shaped, curved, etc.). Tube 505 can include a proximal end 610 and a distal end 615. Further, cannula 520 can exit distal end 615 of tube 505. In some embodiments, cannula 520 can include a pre-bent NiTi cannula which bends when exiting tube 505. Tube 505 and cannula 520 can be constructed of different suitable materials, such as a plastic (e.g, Teflon, Nylon, etc), metal (e.g, Stainless Steal, NiTi, etc), or any other suitable material. Further, in some embodiments, at least one of tube 505 and cannula 520 can rotate about longitudinal axis 620.

In some embodiments, cannula 520 or tube 505 can be used for at least one of drug delivery, aspiration, light delivery, and for delivering at least one of microgrippers, picks, and micro knives. For example, during tele-robotic microsurgery on the eye, a medical professional can extend cannula 520 out of tube 505 into the orbit of the eye. While in the orbit, the medical professional can deliver a micro knife through cannula 520 to remove tissue on the retina.

Further, in some embodiments, cannula 520 can include a backlash-free super-elastic NiTi cannula to provide high precision dexterous manipulation. Using a backlash-free super-elastic NiTi cannula increases the control of delivery into the orbit of the eye by eliminating unwanted movement of the cannula (e.g., backlash). Further, the bending of cannula 520 when exiting tube 505 can increase positioning capabilities for eye surgery.

In some embodiments, the slave hybrid-robot can be designed to manipulate the eye. For example, in some embodiments, at least one of tube 505 and cannula 520 apply force to the eye thereby moving the position of the eye. In some embodiments, force can be applied by cannula 520 inside the eye for manipulating the eye. Force on the eye by at least one of tube 505 and cannula 520 can be generated by moving the parallel robot controlling the position of at least one of the tube and cannula.

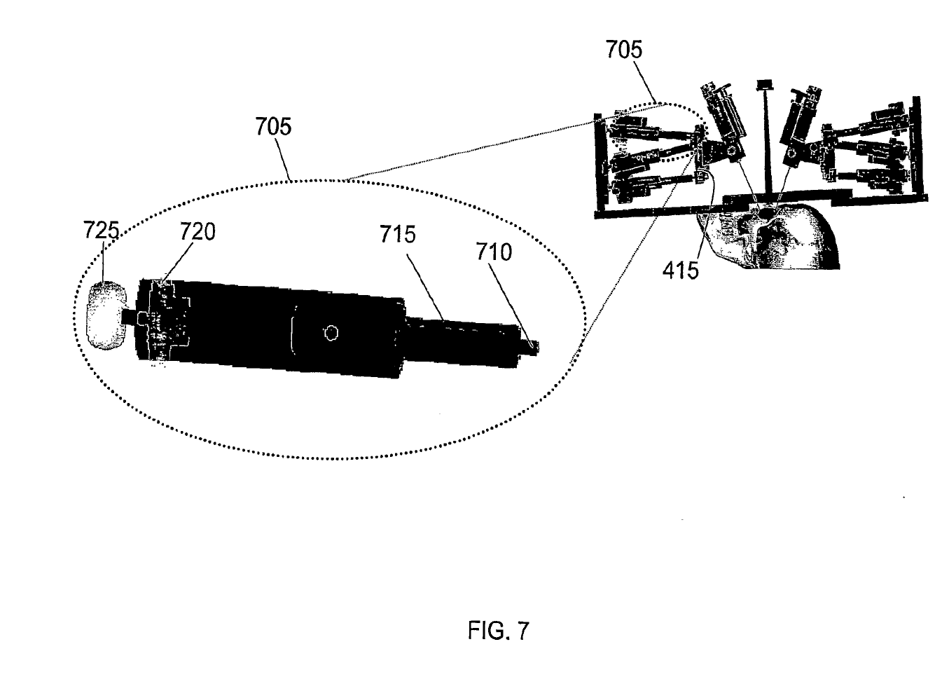

Referring to FIG. 7, the parallel robot can include a plurality of independently actuated legs 705. As the lengths of the independently actuated legs are changed the position and orientation of the platform 415 changes. Legs 705 can include a universal joint 710, a high precision ball screw 715, anti-backlash gear pair 720, and a ball joint 725. The parallel robot can include any number of legs 705. For example, the parallel robot can include three to six legs.

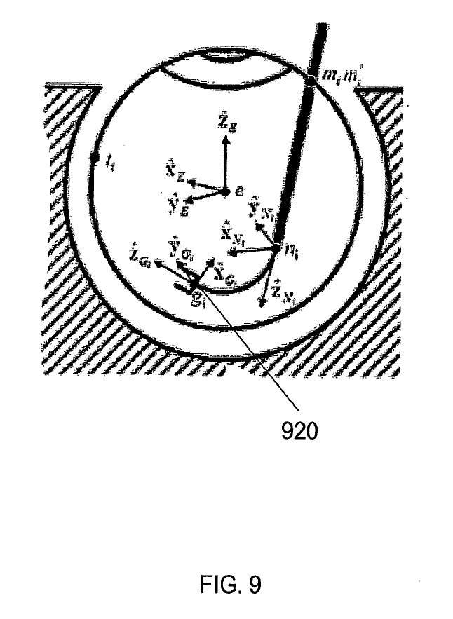

In some embodiments, a unified kinematic model accounts for the relationship between joint speeds (e.g., the speed at which moving parts of the parallel and serial robots translate and rotate) of the two robotic arms of the slave hybrid-robot, and twist of the eye and the surgical tools inside the eye. It will be understood that the twist relates to the six dimensional vector of linear velocity and angular velocity where the linear velocity precedes the angular velocity. The twist can be required to represent the motion of an end effector, described below (920 in FIG. 9). Further, this definition can be different from the standard nomenclature where the angular velocity precedes the linear velocity (in its vector presentation).

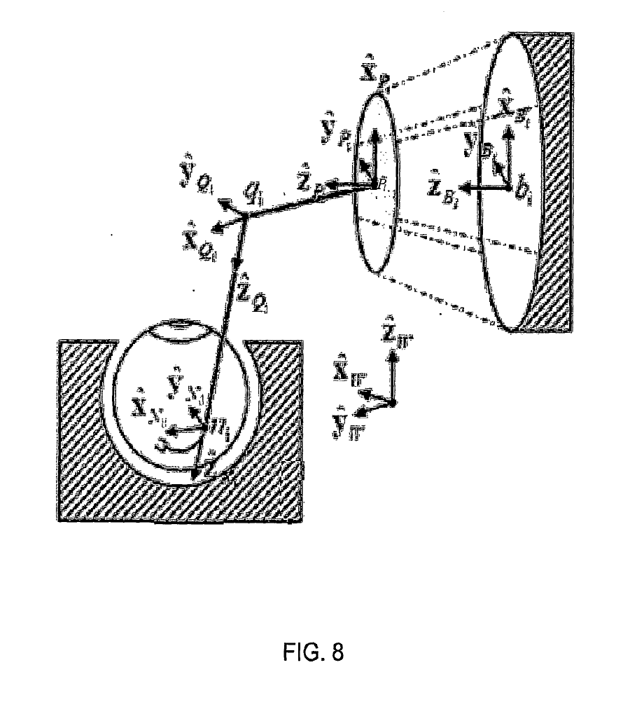

Referring to FIG. 8, the eye and an i^{th} hybrid robot is displayed. The eye system can be enlarged, FIG. 9, for a clearer view of the end effector (e.g., the device at the end of a robotic arm designed to interact with the environment of the eye, such as the pre-bent cannula or items delivered through the pre-bent cannula) and the eye coordinate frames. The coordinate system can be defined to assist in the derivation of the system kinematics. For example, the coordinate systems described below are defined to assist in the derivation of the system kinematics. The world coordinate system {W} (having coordinates \hat{x}_{W}, ŷ_{W}, \hat{z}_{W}) can be centered at an arbitrarily predetermined point in the patient's forehead with the patient in a supine position. The \hat{z}_{W} axis points vertically and ŷ_{W} axis points superiorly (e.g., pointing in the direction of the patients head as viewed from the center of the body along a line parallel to the line formed by the bregma and center point of the foramen magnum of the skull). A parallel robot base coordinate system {Bᵢ} of the i^{th} hybrid robot (having coordinates \hat{x}_{B}_{ᵢ}, ŷ_{B}_{ᵢ}, \hat{z}_{B}_{ᵢ}) can be located at point bᵢ (i.e., the center of the platform base) such that the \hat{z}_{B}_{ᵢ} axis lies perpendicular to the platform base of the parallel robot base and the \hat{x}_{B}_{ᵢ} axis lies parallel to \hat{z}_{W}. The moving platform coordinate system of the i^{th} hybrid robot {Pᵢ} (having coordinates \hat{x}_{P}_{ᵢ}, ŷ_{P}_{ᵢ}, \hat{z}_{P}_{ᵢ}) lies in center of the moving platform, at point pᵢ, such that the axes lie parallel to {Bᵢ} when the parallel platform lies in a home configuration. A parallel extension arm coordinate system of the i^{th} hybrid {Qᵢ} (having coordinates \hat{x}_{Q}_{ᵢ}, ŷ_{Q}_{ᵢ}, \hat{z}_{Q}_{ᵢ}) can be attached to the distal end of the arm at point qᵢ, with \hat{z}_{Q}_{ᵢ} lying along the direction of the insertion needle of the robot, in vector direction q\vec{ᵢn}ᵢ, and \hat{x}_{Q}_{ᵢ} being fixed during setup of eye surgery (e.g., a vitrectomy procedure). The serial robot base coordinate system of the i^{th} hybrid robot {Nᵢ} (having coordinates \hat{x}_{N}_{ᵢ} ŷ_{N}_{ᵢ} \hat{z}_{N}_{ᵢ}) lies at point nᵢ with the \hat{z}_{N}_{ᵢ}, axis also pointing along the insertion needle length of vector q\vec{ᵢn}ᵢ and the ŷ_{N}_{ᵢ} axis rotated from ŷ_{Q}_{ᵢ} an angle qₛ_{ᵢ}₁ about \hat{z}_{N}_{ᵢ}. The end effector coordinator system {Gᵢ} (having coordinates \hat{x}_{G}_{ᵢ}, ŷ_{G}_{ᵢ}, \hat{z}_{G}_{ᵢ}) lies at point gᵢ with the \hat{z}_{G}_{ᵢ} axis pointing in the direction of the end effector gripper 920 and the ŷ_{G}_{ᵢ} can be parallel to the ŷ_{N}_{ᵢ} axis. The eye coordinate system {E} (having coordinates \hat{x}_{E}, ŷ_{E}, \hat{z}_{E}) sits at the center point e of the eye with axes parallel to {W} when the eye is unactuated by the robot.

The notations used are defined below.
i=1,2 refers to an index referring to one of the two arms.
{A} refers to an arbitrary right handed coordinate frame with {\hat{x}_{A}, ŷ_{A}, \hat{z}_{A}} as it is associated unit vectors and point a as the location of its origin.
v^{C}_{A/B}, ω^{C}_{A/B} refers to the relative linear and angular velocities of frame {A} with respect to frame {B}, expressed in frame {C}. Unless specifically stated, all vectors are expressed in {W}.
v_{A}, ω_{A} refers to the absolute linear and angular velocities of frame {A}.
^{A}R_{B} refers to the rotation matrix of the moving frame {B} with respect to the frame {A}.
Rot(\hat{x}_{A}, α) refers to the rotation matrix about unit vector \hat{x}_{A by} an angle α.
[b×] refers to the skew symmetric cross product (i.e., a square matrix A such that it is equal to the negative of its transposed matrix, A=−A^{t}, where superscript t refers to the transpose operator) matrix of b.
\dot{q}_{P}_{ᵢ}=[\dot{q}_{P}_{ᵢ}₁, \dot{q}_{P}_{ᵢ}₂, \dot{q}_{P}_{ᵢ}₃, \dot{q}_{P}_{ᵢ}₄, \dot{q}_{P}_{ᵢ}₅, \dot{q}_{P}_{ᵢ}₆]^{t} refers to the joint speeds of the i^{th} parallel robot platform.
\dot{q}ₛ_{ᵢ}=[\dot{q}ₛ_{ᵢ}₁, \dot{q}ₛ_{ᵢ}_{2]}^{t} refers to the joint speeds of the serial robot. The first component can be the rotation speed about the axis of the serial robot tube and the second component can be the bending angular rate of the pre-bent cannula.
\dot{x}_{A}=[\dot{x}_{A}, \dot{y}_{A}, ż_{A}, ω_{Ax}, ω_{Ay}, ω_{Az}]^{t} refers to the twist of a general coordinate system {A}. For example, referring to FIG. 9A, {Qᵢ} represents the coordinate system defined by its three coordinate axes {\hat{x}_{Q}_{ᵢ}, ŷ_{Q}_{ᵢ}, \hat{z}_{Q}_{ᵢ}}
\dot{x}_{P}_{ᵢ}=[\dot{x}_{P}_{ᵢ}, \dot{y}_{P}_{ᵢ}, ż_{P}_{ᵢ}, ω_{P}_{ᵢ}ₓ, ω_{P}_{ᵢ}_{y}, ω_{P}_{ᵢ}_{z}]^{t} refers to the twist of the moving platform of the i^{th} parallel robot where i=1,2.
\dot{x}ₙ_{ᵢ} refers to the twist of the i^{th} insertion needle end/base of the snake (e.g., the length of the NiTi cannula).
\dot{x}ₑ represents only the angular velocity of the eye (a 3×1 column vector). This is an exception to other notation because it is assumed that the translations of the center of motion of the eye are negligible due to anatomical constraints
^{A}\vec{ab} refers to the vector from point a to b expressed in frame {A}.
r refers to the bending radius of the pre-curved cannula.

$$w ( \vec{a} ) = [ \begin{matrix}{I}_{3 \times 3} & [ - ( \vec{a} ) \times ] \\ {0}_{3 \times 3} & {I}_{3 \times 3}\end{matrix} ]$$

refers to the twist transformation operator. This operator can be defined as a unction of the translation of the origin of the coordinate system indicated by vector \vec{a}. W can be a 6×6 upper triangular matrix with the diagonal elements being a 3×3 unity matrix

$$[ \begin{matrix}100 \\ 010 \\ 001\end{matrix} ]$$

and the upper right 3×3 block being a cross product matrix and the lower left 3×3 block being all zeros.

In some embodiments, the kinematic modeling of the system includes the kinematic constraints due to the incision points in the eye and the limited degrees of freedom of the eye. Below, the kinematics of a two-armed robot with the eye are described, while describing the relative kinematics of a serial robot end effector with respect to a target point on the retina.

The Jacobian of the parallel robot platform, relating the twist of the moving platform frame {Pᵢ} to the joint speeds \dot{q}_{P}_{ᵢ} can be given by:

J_{P}_{ᵢ}\dot{x}_{P}_{ᵢ}=\dot{q}_{P}_{ᵢ} (1)

Developing the next step in the kinematic chain of the i^{th} hybrid robot, to {Qᵢ}, the linear and angular velocities can be expressed with respect to the respective velocities of the moving platform:

v_{Q}_{ᵢ}=v_{P}_{ᵢ}+ω_{P}_{ᵢ}×(\vec{pᵢqᵢ}) (2)

ω_{Q}_{ᵢ}=ω_{P}_{ᵢ} (3)

Writing equations (2) and (3) in matrix form results in the twist of the distal end of the adjustable lockable link:

\dot{x}_{Q}_{ᵢ}=Aᵢ\dot{x}_{P}_{ᵢ} (4)

where Aᵢ=W(\vec{pᵢqᵢ}) can be the twist transformation matrix.

The kinematic relationship of the frame {Nᵢ} can be similarly related to {Qᵢ} by combining the linear and angular velocities. The linear and angular velocities are:

v_{N}_{ᵢ}=v_{Q}_{ᵢ}+ω_{Q}_{ᵢ}×(\vec{qᵢnᵢ}) (5)

ω_{N}_{ᵢ}=ω_{Q}_{ᵢ}+\dot{q}ₛ_{ᵢ}₁\hat{z}_{Q}_{ᵢ} (6)

Equations 5 and 6 expressed in matrix form yield:

$${\dot{x}}_{{N}_{i}} = {B}_{i} {\dot{x}}_{{Q}_{i}} + [ \begin{matrix}0 \\ {\hat{z}}_{{Q}_{i}}\end{matrix} ] {\dot{q}}_{{s}_{i} 1} \qquad ( 7 )$$

where Bᵢ=W(\vec{qᵢnᵢ}).

Continuing to the final serial frame in the hybrid robot, {Gᵢ}, the linear and angular velocities can be written as

v_{G}_{ᵢ}=v_{N}_{ᵢ}+\dot{q}ₛ_{ᵢ}₂r\hat{z}_{G}_{ᵢ}+ω_{N}ᵢ×(\vec{nᵢgᵢ}) (8)

ω_{G}_{ᵢ}=ω_{N}_{ᵢ}+\dot{q}ₛ_{ᵢ}₂ŷ_{N}_{ᵢ} (9)

Equations 8 and 9 expressed in matrix form yield:

$${\dot{x}}_{{G}_{i}} = {C}_{i} {\dot{x}}_{{N}_{i}} + [ \begin{matrix}r {\hat{z}}_{{G}_{i}} \\ {\hat{y}}_{{N}_{i}}\end{matrix} ] {\dot{q}}_{{s}_{i} 2} \qquad ( 10 )$$

where Cᵢ=W(\vec{nᵢgᵢ}).

To express the kinematics of the frame of the robot end effector, {Gᵢ}, as a function of the joint parameters of the i^{th} hybrid robotic system, the serial relationships developed above can be combined. Beginning with the relationship between the twist of frame {Gᵢ} and {Nᵢ} and inserting the relationship between {Nᵢ} and {Qᵢ} yields:

$${\dot{x}}_{{G}_{i}} = {C}_{i} {B}_{i} {\dot{x}}_{{Q}_{i}} + {C}_{i} [ \begin{matrix}0 \\ {\hat{z}}_{{Q}_{i}}\end{matrix} ] {\dot{q}}_{{s}_{i} 1} + [ \begin{matrix}r {\hat{z}}_{{G}_{i}} \\ {\hat{y}}_{{N}_{i}}\end{matrix} ] {\dot{q}}_{{s}_{i} 2} \qquad ( 11 )$$

Further, by reintroducing the matrix Cᵢ to the \dot{q}_{s,1} term, the serial joints of the hybrid system can be parameterized as follows:

\dot{x}_{G}_{ᵢ}=CᵢBᵢ\dot{x}_{Q}_{ᵢ}+Jₛ_{ₓ}\dot{q}ₛ_{ᵢ} (12)

where

$${J}_{{s}_{i}} = [ \begin{matrix}[ ( - \vec{{n}_{i} {g}_{i}} ) \times ] {\hat{z}}_{{Q}_{i}} & r {\hat{z}}_{{G}_{i}} \\ {\hat{z}}_{{Q}_{i}} & {\hat{y}}_{{N}_{i}}\end{matrix} ]$$

represents the Jacobian of the serial robot including the speeds of rotation about the axis of the serial robot cannula and the bending of the pre-curved cannula.

Inserting the relationship between {Qᵢ} and {Pᵢ} and the inverse of the Stewart Jacobian equation (1), and condensing terms yields the final Jacobian for the i^{th} hybrid robot yields:

\dot{x}_{G}_{ᵢ}=Jₕ_{ᵢ}\dot{q}ₕ_{ᵢ} (13)

where Jₕ_{ᵢ}=└CᵢBᵢAᵢJ_{P}_{ᵢ}⁻¹, Jₛ_{ᵢ}┘.

The eye can be modeled as a rigid body constrained to spherical motion by the geometry of the orbit and musculature. Roll-Pitch-Yaw angles (α,β,γ) can be chosen to describe the orientation of the eye such that the rotation matrix ^{w}Rₑ specifies the eye frame {E} with respect to {W} as ^{W}Rₑ=R_{z}R_{y}Rₓ where Rₓ=Rot(\hat{x}_{W},α), R_{y}=Rot(ŷ_{W},β), and R_{z}=Rot(\hat{z}_{W},γ).

The angular velocity of the eye can be parameterized by:

\dot{x}ₑ=[\dot{α},\dot{β},\dot{γ}]^{t} (4)

The kinematics of the end effector with respect to the eye can also be modeled. For example, with the kinematics of the eye and the i^{th} hybrid robotic system characterized separately, the formulations can be combined to define the kinematic structure of the eye and i^{th} hybrid robot. This relationship can allow expression of the robot joint parameters based on the desired velocity of the end effector with respect to the eye and the desired angular velocity of the eye. To achieve this relationship, an arbitrary goal point on the retinal surface tᵢ can be chosen. The angular velocity of the eye imparts a velocity at point tᵢ

vₜ_{ᵢ}=Tᵢ\dot{x}ₑ (15)

where end effector Tᵢ=└(−\vec{et}ᵢ)×┘

The linear velocity of the end effector frame of the robot with respect to the goal point tᵢ can be written as:

v_{g}_{ᵢ}_{/t}_{ᵢ}=v_{g}_{ᵢ}−vₜ_{ᵢ} (16)

Inserting equations (13) and equations (15) into equation (16) yields a linear velocity of the end effector as a function of the robot joint speeds and the desired eye velocity

v_{g}_{ₜ}_{/t}_{ᵢ}=[I_{3×3},0_{3×3}]Jₕ_{ᵢ}\dot{q}ₕ_{ᵢ}−Tᵢ\dot{x}ₑ (17)

Similarly, the angular velocity of the end effector frame of the robot with respect to the eye frame can be written as:

ω_{g}_{ₜ}_{/e}=ω_{g}_{ᵢ}−ωₑ (18)

or, by inserting equation (13) and equation (15) into equation (18) yielding

ω_{g}_{ᵢ}_{/e}=[0_{3×3},I_{3×3}]Jₕ_{ᵢ}\dot{q}ₕ_{ᵢ}−\dot{x}ₑ (19)

further combining the linear equation (17) and angular equation (19) velocities yields the twist of the end effector with respect to point tᵢ:

\dot{x}_{g}_{ᵢ}_{/t}_{ᵢ}=Jₕ_{ᵢ}\dot{q}ₕ_{ᵢ}−Dᵢ\dot{x}ₑ (20)

where Dᵢ=[Tᵢ^{t},I_{3×3}]^{t}.

In some embodiments, the mechanical structure of the hybrid robot in the eye (e.g., vitreous cavity) allows only five degrees of freedom as independent rotation about the \hat{z}_{G}_{ᵢ} axis can be unachievable. This rotation can be easily represented by the third w-v-w Euler angle φᵢ. It should be noted that the first angle φᵢ represents the rotation between the projection of the \hat{z}_{G}_{ᵢ} axis on the \hat{x}_{W}ŷ_{W} plane and \hat{x}_{W} and the second angle θᵢ represents rotation between \hat{z}_{W} and \hat{z}_{G}_{ᵢ}.

The system can utilize path planning and path control. For example, path planning and path control can be used to ease the surgery by having the tele-robotic master controller automatically perform some of the movements for the slave hybrid-robot. For the purposes of path planning and control, the twist of the system can therefore be parameterized with w-v-w Euler angles and the third Euler angle eliminated by a degenerate matrix Kᵢ defined as follows:

{dot over (\tilde{x}_{g}_{ᵢ}_{/t}_{ᵢ}=Kᵢ\dot{x}_{g}_{ᵢ}_{/t}_{ᵢ} (21)

Inserting this new parameterization into the end effector twist yields a relation between the achievable independent velocities and the joint parameters of the hybrid system.

[symbol]

+KᵢDᵢ\dot{x}ₑ=KᵢJₕ_{ᵢ}\dot{q}ₕ_{ᵢ} (22)

The robotic system can be constrained such that the hybrid robots move in concert (e.g., move substantially together) to control the eye without injuring the structure by tearing the insertion points. This motion can be achieved by allowing each insertion arm to move at the insertion point only with the velocity equal to the eye surface at that point, plus any velocity along the insertion needle. This combined motion constrains the insertion needle to the insertion point without damage to the structure.

To assist in the development of the aforementioned constraint, point mᵢ can be defined at the insertion point on the scleral surface of the eye and m′ᵢ can be defined as point on the insertion needle instantaneously coincident with mᵢ. To meet the above constraint, the velocity of mᵢ must be equal to the velocity of point mᵢ in the plane perpendicular to the needle axis:

v_{m′}_{ᵢ}_{⊥}=vₘ_{ᵢ}_{⊥} (23)

Taking a dot product in the directions, \hat{x}_{Q}_{ᵢ} and ŷ_{Q}_{ᵢ} yields two independent constraint equations:

\hat{x}_{Q}_{ᵢ}^{t}v_{m′}_{ᵢ}=\hat{x}_{Q}_{ᵢ}^{t}vₘ_{ᵢ} (24)

ŷ_{Q}_{ᵢ}^{t}v_{m′}_{ᵢ}=ŷ_{Q}_{ᵢ}^{t}vₘ_{ᵢ} (25)

These constraints can be expressed in terms of the joint angles by relating the velocities of point mᵢ and m′ᵢ to the robot coordinate systems. The velocity of point m; can be related to the velocity of frame {Qᵢ} as follows:

v_{m′}_{ᵢ}=v_{Q}_{ᵢ}+ω_{Q}_{ᵢ}×\vec{qᵢm}ᵢ (26)

By substituting the twist of frame {Qᵢ}, the above equation becomes:

v_{m′}_{ᵢ}=[I_{3×3}, 0_{3×3}]\dot{x}_{Q}_{ᵢ}+Eᵢ[0_{3×3}, I_{3×3}]\dot{x}_{Q}_{ᵢ} (27)

where Eᵢ=[\vec{qᵢmᵢ}×].

Inserting equations (4) and (1) and writing in terms of the hybrid joint parameters \dot{q}ₕ_{ᵢ} yields:

v_{m′}_{ᵢ}=Fᵢ\dot{q}ₕ_{ᵢ} (28)

where Fᵢ=([I_{3×3},0_{3×3}]−Eᵢ[0_{3×3},I_{3×3}])AᵢJ_{P}_{ᵢ}⁻¹[I_{6×6},0_{6×2}].

An expression for the velocity of the insertion point mᵢ can be related to the desired eye velocity, similar to the derivation of velocity of point tᵢ, yielding:

vₘ_{ᵢ}=Mᵢ\dot{x}ₑ (29)

where Mᵢ=└(−\vec{emᵢ})×┘.

Substituting equation (28) and equation (29) into equation (24) and equation (25) yields the final constraint equations given for the rigid body motion of the eye-robot system:

\hat{x}_{Q}_{ᵢ}^{t}Fᵢ\dot{q}ₕ_{ᵢ}=\hat{x}_{Q}_{ᵢ}^{t}Mᵢ\hat{x}ₑ (30)

ŷ_{Q}_{ᵢ}^{t}Fᵢ\dot{q}ₕ_{ᵢ}=ŷ_{Q}_{ᵢ}^{t}Mᵢ\dot{x}ₑ (31)

Combining these constraints with the twist of the hybrid systems for indices 1 and 2, yields the desired expression of the overall eye-robotic system relating the hybrid robotic joint parameters to the desired end effector twists and the desired eye velocity.

$$[ \begin{matrix}{K}_{1} {J}_{{h}_{1}} & {0}_{5 \times 8} \\ {0}_{5 \times 8} & {K}_{2} {J}_{{h}_{2}} \\ {G}_{1} {F}_{1} & {0}_{2 \times 8} \\ {0}_{2 \times 8} & {G}_{2} {F}_{2}\end{matrix} ] [ \begin{matrix}{\dot{q}}_{{h}_{1}} \\ {\dot{q}}_{{h}_{2}}\end{matrix} ] = [ \begin{matrix}{I}_{5 \times 5} & {0}_{5 \times 5} & {K}_{1} {D}_{1} \\ {0}_{5 \times 5} & {I}_{5 \times 5} & {K}_{2} {D}_{2} \\ {0}_{2 \times 5} & {0}_{2 \times 5} & {G}_{1} {M}_{1} \\ {0}_{2 \times 5} & {0}_{2 \times 5} & {G}_{2} {M}_{2}\end{matrix} ] [ \begin{matrix}{\tilde{\dot{x}}}_{{g}_{1} / {t}_{1}} \\ {\tilde{\dot{x}}}_{{g}_{2} / {t}_{2}} \\ {\dot{x}}_{e}\end{matrix} ] \qquad ( 32 )$$

where Gᵢ=[\hat{x}_{O}_{₁},ŷ_{O}_{₁}]

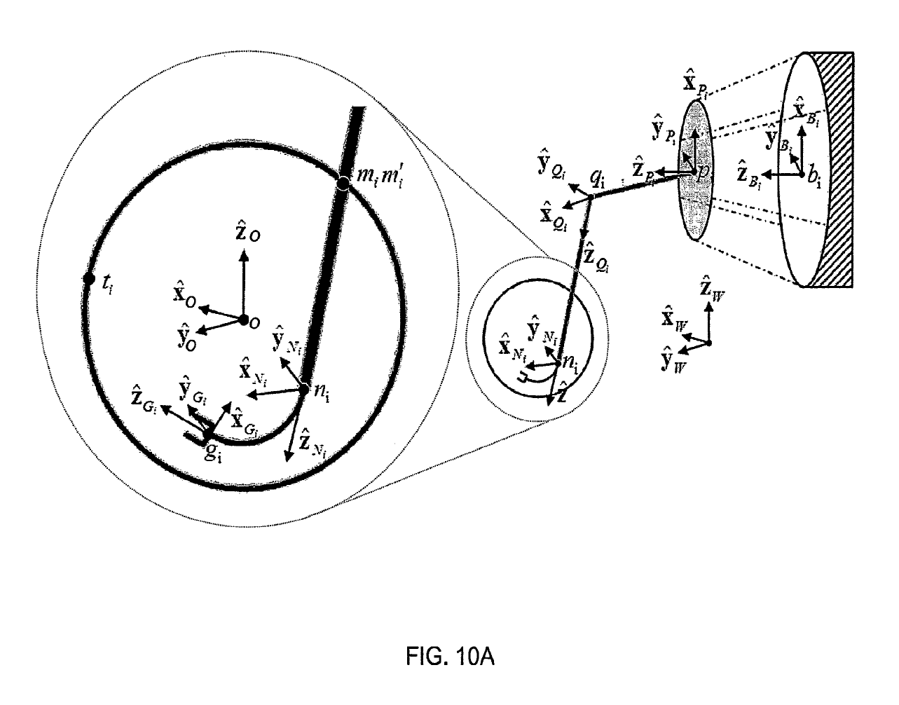

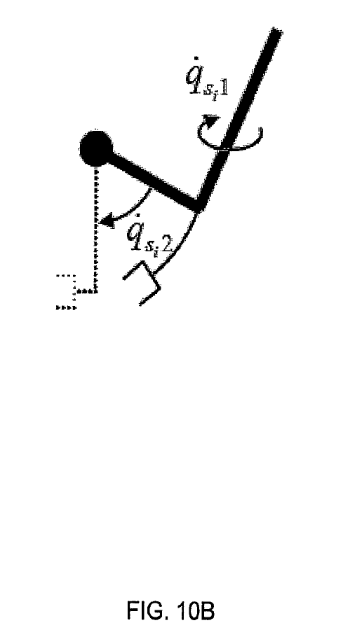

Referring to FIG. 10A-10B, an organ and the i^{th} hybrid robotic arm is displayed. The organ is enlarged (FIG. 10A) for a clearer view of the end effector and the organ coordinate frames. FIG. 10B illustratively displays an enlarged view of the end effector. The following coordinate systems are defined to assist in the derivation of the system kinematics. The world coordinate system {W} (having coordinates \hat{x}_{W}, ŷ_{W}, \hat{z}_{W}) can be centered at an arbitrarily predetermined point in the patient's forehead with the patient in a supine position. The \hat{z}_{W} axis points vertically and ŷ_{W} axis points superiorly. The parallel robot base coordinate system {Bᵢ} (having coordinates \hat{x}_{B}_{ᵢ}, ŷ_{B}_{ᵢ}, \hat{z}_{B}_{ᵢ}) of the i^{th} hybrid robot can be located at point bᵢ (i.e., the center of the base platform) such that the \hat{z}_{B}_{ᵢ} axis lies perpendicular to the base of the parallel robot platform and the \hat{x}_{B}_{ᵢ} axis lies parallel to \hat{z}_{W}. The moving platform coordinate system of the i^{th} hybrid robot {Pᵢ} (having coordinates \hat{x}_{P}_{ᵢ}, ŷ_{P}_{ᵢ}, \hat{z}_{P}_{ᵢ}) lies in center of the moving platform, at point pᵢ such that the axes lie parallel to {Bᵢ} when the parallel robot platform lies in the home configuration (e.g., the initial setup position). The parallel robot extension arm coordinate system of the i^{th} hybrid {Qᵢ} (having coordinates \hat{x}_{Q}_{ᵢ}, ŷ_{Q}_{ᵢ}, \hat{z}_{Q}_{ᵢ}) can be attached to the distal end of the arm at point qᵢ, with \hat{z}_{Q}_{ᵢ} lying along the direction of the insertion needle of the robot \vec{qᵢnᵢ}, and \hat{x}_{Q}_{ᵢ} fixed during setup procedure. The serial robot (e.g., intra-ocular dexterity robot) base coordinate system of the i^{th} hybrid robot {Nᵢ} (having coordinates \hat{x}_{N}_{ᵢ} ŷ_{N}_{ᵢ} \hat{z}_{N}_{ᵢ}) lies at point nᵢ with the \hat{z}_{N}_{ᵢ} axis also pointing along the insertion needle length \vec{qᵢnᵢ} and the ŷ_{N}_{ᵢ} axis rotated from ŷ_{Q}_{ᵢ} an angle qₛ_{ᵢ}₁ about \hat{z}_{N}_{ᵢ}. The end effector coordinate system {Gᵢ} (having coordinates \hat{x}_{G}_{ᵢ}, ŷ_{G}_{ᵢ}, \hat{z}_{G}_{ᵢ}) lies at point gᵢ with the \hat{z}_{G}ᵢ, axis pointing in the direction of the end effector gripper and the ŷ_{G}_{ᵢ} axis parallel to the ŷ_{N}_{ᵢ} axis. The organ coordinate system {O} (having coordinates \hat{x}_{O}, ŷ_{O}, \hat{z}_{O}) sits at the rotating center o of the organ with axes parallel to {W} when the organ can be not actuated by the robot.

The additional notations used are defined below:
i refers to the index identifying each robotic arm. Further, for unconstrained organs i=1,2,3 while for the eye i=1,2.
{A} refers to a right handed coordinate frame with {\hat{x}_{A},ŷ_{A},\hat{z}_{A}} as its associated unit vectors and point a as the location of its origin.
v^{C}_{A/B}, ω^{C}_{A/B} refers to the relative linear and angular velocities of frame {A} with respect to {B}, expressed in {C}. It will be understood that, unless specifically stated, all vectors displayed below can be expressed in {W}.
v_{A},ω_{A} refers to absolute linear and angular velocities of frame {A}.
^{A}R_{B} refers to the rotation matrix of the moving frame {B} with respect to {A}.
Rot(\hat{x}_{A}, α) refers to the rotation matrix about unit vector \hat{x}_{A} by angle α.
[b×] refers to the skew symmetric cross product matrix of vector b.
\dot{q}_{P}_{ᵢ}=[\dot{q}_{P}_{ᵢ}₁,\dot{q}_{P}_{ᵢ}₂,\dot{q}_{P}_{ᵢ}₃,\dot{q}_{P}_{ᵢ}₄,\dot{q}_{P}_{ᵢ}₅,\dot{q}_{P}_{ᵢ}₆]^{t} refers to the active joint speeds of the i^{th} parallel robot platform.
\dot{q}ₛ_{ᵢ}=[\dot{q}ₛ_{ᵢ}₁,\dot{q}ₛ_{ᵢ}₂]^{t} refers to the joint speeds of the i^{th} serial robot (e.g., intra-ocular dexterity robot). The first component can be the rotation speed about the axis of the serial robot (e.g., intra-ocular dexterity robot) tube, and the second component can be the bending angular rate of the pre-shaped cannula.
\dot{x}_{A}, \dot{x}_{P}_{ᵢ}, \dot{x}ₒ refers to the twists of frame {A}, of the i^{th} parallel robot moving platform, and of the organ.
^{A}\vec{ab} refers to the vector from point a to b expressed in frame {A}.
Lₛ refers to the bending radius of the pre-bent cannula of the serial robot (e.g., intra-ocular dexterity robot).

$$w ( \vec{a} ) = [ \begin{matrix}{I}_{3 \times 3} & [ - ( \vec{a} ) \times ] \\ {0}_{3 \times 3} & {I}_{3 \times 3}\end{matrix} ]$$

refers to the twist transformation operator. This operator can be defined as a function of the translation of the origin of the coordinate system indicated by vector \dot{a}. W can be a 6×6 upper triangular matrix with the

$$[ \begin{matrix}100 \\ 010 \\ 001\end{matrix} ]$$

diagonal elements being a 3×3 unity matrix and the upper right 3×3 block being a cross product matrix and the lower left 3×3 block being all zeros.

In some embodiments, the kinematic modeling of the system can include the kinematic constraints of the incision points on the hollow organ. Below, the kinematics of the triple-armed robot with the organ and describes the relative kinematics of the serial robot (e.g., intra-ocular dexterity robot) end effector with respect to a target point on the organ.

The Jacobian of the parallel robot platform relating the twist of the moving platform frame \dot{x}ₚ_{ᵢ} to the joint parameters, \dot{q}ₚ_{ᵢ} is shown in equation 33. Further, the overall hybrid Jacobian matrix for one robotic arm is obtained as equation 34.

J_{P}_{ᵢ}\dot{x}_{P}_{ᵢ}=\dot{q}_{P}_{ᵢ} (33)

\dot{x}_{G}_{ᵢ}=Jₕ_{ᵢ}\dot{q}ₕ_{ᵢ} (34)

In some embodiments, modeling can be accomplished by considering the elasticity and surrounding anatomy of the organ. Further, in some embodiments, the below analysis does not include the organ elasticity. Further still, a six dimension twist vector can be used to describe the motion of the organ using the following parameterization:

\dot{x}ₒ=[\dot{x}ₒₗ^{t},\dot{x}ₒₙ^{t}]^{t}=[\dot{x},\dot{y},ż, \dot{α},\dot{β},\dot{γ}]^{t} (35)

where x,y,z,α,β,γ can be linear positions and Roll-Pitch-Yaw angles of the organ, and \dot{x}ₒₗ and \dot{x}ₒₙ correspond to the linear and angular velocities of the organ respectively.

In some embodiments, the Kinematics of the serial robot (e.g., intra-ocular dexterity robot) end effector with respect to the organ can be modeled. Further, in some embodiments, the model can express the desired velocity of the end effector with respect to the organ and the desired velocity of the organ itself, an arbitrary target point tᵢ on the inner surface of the organ can be chosen. The linear and angular velocities of the end effector frame with respect to the target point can be written as:

v_{g}_{ᵢ}_{/t}_{ᵢ}=[I_{3×3},0_{3×3}]Jₕ_{ᵢ}\dot{q}ₕ_{ᵢ}−\dot{x}ₒₗ−Tᵢ\dot{x}ₒₙ (36)

ω_{g}_{ᵢ}_{/o}=[0_{3×3},I_{3×3}]Jₕ_{ᵢ}\dot{q}ₕ_{ᵢ}−\dot{x}ₒₙ (37)

Further, combining equation 36 and equation 37 yields the twist of the end effector with respect to point tᵢ:

\dot{x}_{g}_{ᵢ}_{/t}_{ᵢ}=Jₕ_{ᵢ}\dot{q}ₕ_{ᵢ}−Hᵢ\dot{x}ₒ (38)

where Tᵢ=└(−\vec{otᵢ})×┘ and

$${H}_{i} = [ \begin{matrix}{I}_{3 \times 3} & {T}_{i} \\ {0}_{3 \times 3} & {I}_{3 \times 3}\end{matrix} ]$$

The mechanical structure of the hybrid robot in the organ cavity can allow only five degrees of freedom as independent rotation of the serial robot (e.g., intra-ocular dexterity robot) end effector about the \hat{z}_{G}_{ᵢ} axis can be unachievable due to the two degrees of freedom of the serial robot (e.g., intra-ocular dexterity robot). This rotation can be represented by the third w-v-w Euler angle φᵢ. In some embodiments, for the purposes of path planning and control, the twist of the system can be parameterized using w-v-w Euler angles while eliminating the third Euler angle through the use of a degenerate matrix Kᵢ as defined below. Inserting the aforementioned parameterization into the end effector twist, equation 38, yields a relation between the achievable independent velocities and the joint parameters of the hybrid system, equation 40.

[symbol]

=Kᵢ\dot{x}_{g}_{ᵢ}_{/t}_{ᵢ} (39)

[symbol]

+KᵢHᵢ\dot{x}ₒ=KᵢJₕ_{ᵢ}\dot{q}ₕ_{ᵢ} (40)

In some embodiments, the robotic system can be constrained such that the hybrid arms move synchronously to control the organ without tearing the insertion point. For example, the robotic system can be constrained such that the multitude, nₐ, of hybrid robotic arms moves synchronously to control the organ without tearing the insertion points. The i^{th} incision point on the organ be designated by point mᵢ, i=1,2,3 . . . nₐ. The corresponding point, which can be on the serial robot (e.g., intra-ocular dexterity robot) cannula of the i^{th} robotic arm and instantaneously coincident with mᵢ, be designated by m′ᵢ, i=1,2,3 . . . nₐ. In some embodiments, to prevent damage to the anatomy, an equality constraint must be imposed between the projections of the linear velocities of mᵢ and m′ᵢ on a plane perpendicular to the longitudinal axis of the i^{th} serial robot (e.g., intra-ocular dexterity robot) cannula. These conditions can be given in equation 41 and equation 42 as derived in detail below.

\hat{x}_{Q}_{ᵢ}^{t}Fᵢ\dot{q}ₕ_{ᵢ}=\hat{x}_{Q}_{ᵢ}^{t}(\dot{x}ₒₗ+Mᵢ\dot{x}ₒₙ), i=1, 2, 3 . . . nₐ (41)

ŷ_{Q}_{ᵢ}^{t}Fᵢ\dot{q}ₕ_{ᵢ}=ŷ_{Q}_{ᵢ}^{t}(\dot{x}ₒₗ+Mᵢ\dot{x}ₒₙ), i=1, 2, 3 . . . nₐ (42)

Equation 41 and equation 42 can constitute 2nₐ scalar equations that provide the conditions for the organ to be constrained by nₐ robotic arms inserted into it through incision points. For the organ to be fully constrained by the robotic arms, equation 41 and equation 42 should have the same rank as the dimension of the organ twist, \dot{x}ₒ as constrained by its surrounding anatomy. Further, if the organ is a free-floating organ, then the rank should be six and therefore a minimum of three robotic arms can be necessary to effectively stabilize the organ. Further still, if the organ is constrained from translation (e.g., as for the eye), the required rank can be three and hence the minimum number of arms can be two (e.g., for a dual-arm ophthalmic surgical system).

Combining the constraint equations as derived below with the twist of the hybrid robotic arms [symbol] for i=1, 2, 3, yields the desired expression of the overall organ-robotic system relating the joint parameters of each hybrid robotic arm to the desired end effector twists and to the organ twist.

$$[ \begin{matrix}\underbrace{\begin{matrix}{K}_{1} {J}_{{h}_{1}} & {0}_{5 \times 8} & {0}_{5 \times 8} \\ {0}_{5 \times 8} & {K}_{2} {J}_{{h}_{2}} & {0}_{5 \times 8} \\ {0}_{5 \times 8} & {0}_{5 \times 8} & {K}_{3} {J}_{{h}_{3}} \\ {G}_{1} {F}_{1} & {0}_{2 \times 8} & {0}_{2 \times 8} \\ {0}_{2 \times 8} & {G}_{2} {F}_{2} & {0}_{2 \times 8} \\ {0}_{2 \times 8} & {0}_{2 \times 8} & {G}_{3} {F}_{3}\end{matrix}} \\ {J}_{I}\end{matrix} ] [ \begin{matrix}{\dot{q}}_{{h}_{1}} \\ {\dot{q}}_{{h}_{2}} \\ {\dot{q}}_{{h}_{3}}\end{matrix} ] = [ \begin{matrix}\underbrace{\begin{matrix}{I}_{5 \times 5} & {0}_{5 \times 5} & \begin{matrix}{0}_{5 \times 5} & {K}_{1} {H}_{1}\end{matrix} \\ {0}_{5 \times 5} & {I}_{5 \times 5} & \begin{matrix}{0}_{5 \times 5} & {K}_{2} {H}_{2}\end{matrix} \\ {0}_{5 \times 5} & {0}_{5 \times 5} & \begin{matrix}{1}_{5 \times 5} & {K}_{3} {H}_{3}\end{matrix} \\ {0}_{2 \times 5} & {0}_{2 \times 5} & \begin{matrix}{0}_{2 \times 5} & {G}_{1} {P}_{1}\end{matrix} \\ {0}_{2 \times 5} & {0}_{2 \times 5} & \begin{matrix}{0}_{2 \times 5} & {G}_{2} {P}_{2}\end{matrix} \\ {0}_{2 \times 5} & {0}_{2 \times 5} & \begin{matrix}{0}_{2 \times 5} & {G}_{3} {P}_{3}\end{matrix}\end{matrix}} \\ {J}_{O}\end{matrix} ] [ \begin{matrix}{\tilde{\dot{x}}}_{{g}_{1} / {t}_{1}} \\ {\tilde{\dot{x}}}_{{g}_{2} / {t}_{2}} \\ {\tilde{\dot{x}}}_{{g}_{3} / {t}_{3}} \\ {\dot{x}}_{o}\end{matrix} ] \qquad ( 43 )$$

Considering the contact between fingers (e.g., graspers delivered into an organ) and the payload (e.g., the organ) a differential kinematic relationship can be modeled. Further, multi-arm manipulation can be modeled wherein the relative position between the robotic arms and the organ can be always changing. Further, by separating input joint rates \dot{q}ₕ output organ motion rates \dot{x}ₒ and relative motion rates [symbol] equation 43, the kinematic relationship can be modeled.

The robot kinetostatic performance can be evaluated by examining the characteristics of the robot Jacobian matrix. Further, normalization of the Jacobian can be necessary when calculating the singular values of the Jacobian. These singular values can depend on the units of the individual cells of the Jacobian. Inhomogeneity of the units of the Jacobian can stem from the inhomogeneity of the units of its end effector twist and inhomogeneity of the units in joint space (e.g., in cases where not all the joints are of the same type, such as linear or angular). Normalizing the Jacobian matrix requires scaling matrices corresponding to ranges of joint and task-space variables by multiplying the Jacobian for normalization. Further, using the characteristic length to normalize the portion of the Jacobian bearing the unit of length and using a kinematic conditioning index defined as the ratio of the smallest and largest singular value of a normalized Jacobian the performance can be evaluated. Further still, the Jacobian scaling matrix can be found by using a physically meaningful transformation of the end effector twist that would homogenize the units of the transformed twist. The designer can be required to determine the scaling/normalization factors of the Jacobian prior to the calculation of the condition index of the Jacobian. The methodology used relies on the use of individual characteristic lengths for the serial and the parallel portions of each robotic arm.

Equations 44-46 specify the units of the individual vectors and submatrices of equation 43. The brackets can be used to designate units of a vector or a matrix, where [m] and [s] denote meters and seconds respectively. The Jacobian matrices Jₗ and Jₒ do not possess uniform units and using a single characteristic length to normalize both of them can be not possible because the robotic arms can include both serial and parallel portions. Also, evaluating the performance of the robotic system for different applications can include simultaneously normalizing Jₗ and Jₒ rendering the units of all their elements to be unity. Further, this can be achieved through an inspection of the units of these matrices and the physical meaning of each submatrix in equation 43 while relating each matrix block to the kinematics of the parallel robot, or the serial robot (e.g., intra-ocular dexterity robot), or the organ.

$$\begin{matrix}[ {\tilde{\dot{x}}}_{{g}_{i} / {t}_{i}} ] = {[ {[ m / s ]}_{1 \times 3} , {[ 1 / s ]}_{1 \times 2} ]}^{t} , \\ [ {\dot{x}}_{o} ] = {[ {{[ m / s ]}_{1 \times 3} [ 1 / s ]}_{1 \times 3} ]}^{t} \\ ⌊ {\dot{q}}_{{h}_{i}} ⌋ = {[ {[ m / s ]}_{1 \times 6} , {[ 1 / s ]}_{1 \times 2} ]}^{t} & ( 44 ) \\ [ {G}_{i} {P}_{i} ] = [ {{[ 1 ]}_{2 \times 3} [ m ]}_{2 \times 3} ] , [ {G}_{i} {F}_{i} ] = [ {{[ 1 ]}_{2 \times 6} [ 0 ]}_{2 \times 2} ] & ( 45 ) \\ [ {K}_{i} {H}_{i} ] = [ \begin{matrix}{[ 1 ]}_{3 \times 3} & {[ m ]}_{3 \times 3} \\ {[ 0 ]}_{2 \times 3} & {[ 1 ]}_{2 \times 3}\end{matrix} ] , [ {K}_{i} {J}_{{h}_{i}} ] = [ \begin{matrix}{[ 1 ]}_{3 \times 6} & {[ m ]}_{3 \times 2} \\ {[ 1 / m ]}_{2 \times 6} & {[ 1 ]}_{2 \times 2}\end{matrix} ] & ( 46 )\end{matrix}$$

When the Jacobian matrix J_{O} characterizes the velocities of the rotating organ and the end effector, the matrix can be homogenized using the radius of the organ at the target point as the characteristic length. It can be this radius, as measured with respect to the instantaneous center of rotation that imparts a linear velocity to point tᵢ, as a result of the angular velocity of the organ. The top right nine components of J_{O} given by KᵢHᵢ i=1,2,3 of equation 43, bear the unit of [m]. Hence, dividing them by the radius of the organ at the target point, Lᵣ can render their units to be unity. The same treatment can be also carried out to the rightmost six components of each matrix block GᵢPᵢ i=1,2,3, where we divide them by Lᵣ as well.

The Jacobian matrix Jₗ can describe the geometry of both the parallel robot and the serial robot. Further this can be done by using both Lₚ, the length of the connection link of the parallel robot, \vec{pᵢqᵢ}, and Lₛ, the bending radius of the inner bending tube of the serial robot, as characteristic lengths. In some instances, Lₚ is multiplied by those components in KᵢJₕ_{ᵢ} bearing the unit of [1/m]. Further, the components in KᵢJₕ_{ᵢ} that bear the unit of [m] can be divided by Lₛ. This can result in a normalized input Jacobian Jₗ that can be dimensionless. Further still, the radius of the moving platform can be used for normalization. Lₚ can be the scaling factor of the linear velocity at point qᵢ stemming from a unit angular velocity of the moving platform. Similarly, the circular bending cannula of the serial robot can be modeled as a virtual rotary joint, and the bending radius Lₛ can be used to normalize the components of KᵢJₕ_{ᵢ} that are related to the serial robot.

In some embodiments, the eye can be modeled as a constrained organ allowing only rotational motions about its center. This can be used to produce a simplify model of the twist of the organ as a three dimensional vector as indicated in equation 47. The relative linear and angular velocities of the robot arm end effector with respect to a target point t; on the retina are given by equation 48 and equation 49, which can be combined to yield the relative twist between the end effector of each arm, and the target point, equation 50, where Dᵢ=[Tᵢ^{t},I_{3×3}]^{t} while the five dimensional constrained twist of the serial robot end effector in equation 40 simplifies to equation 51. Further, the overall Jacobian equation for the whole system with the eye simplifies to equation 52.

$$\begin{matrix}{\dot{x}}_{e} = {[ \dot{α} , \dot{β} , \dot{γ} ]}^{t} & ( 47 ) \\ {v}_{{g}_{i} / {t}_{i}} = [ {I}_{3 \times 3} , {0}_{3 \times 3} ] {J}_{{h}_{i}} {\dot{q}}_{{h}_{i}} - {T}_{i} {\dot{x}}_{e} & ( 48 ) \\ {ω}_{{g}_{i} / e} = [ {0}_{3 \times 3} , {I}_{3 \times 3} ] {J}_{{h}_{i}} {\dot{q}}_{{h}_{i}} - {\dot{x}}_{e} & ( 49 ) \\ {\dot{x}}_{{g}_{i} / {t}_{i}} = {J}_{{h}_{i}} {\dot{q}}_{{h}_{i}} - {D}_{i} {\dot{x}}_{e} & ( 50 ) \\ {\tilde{\dot{x}}}_{{g}_{i} / {t}_{i}} + {K}_{i} {D}_{i} {\dot{x}}_{e} = {K}_{i} {J}_{{h}_{i}} {\dot{q}}_{{h}_{i}} & ( 51 ) \\ \underbrace{[ \begin{matrix}{K}_{1} {J}_{{h}_{1}} & {0}_{5 \times 8} \\ {0}_{5 \times 8} & {K}_{2} {J}_{{h}_{2}} \\ {G}_{1} {F}_{1} & {0}_{2 \times 8} \\ {0}_{2 \times 8} & {G}_{2} {F}_{2}\end{matrix} ]}_{M} [ \begin{matrix}{\dot{q}}_{{h}_{1}} \\ {\dot{q}}_{{h}_{2}}\end{matrix} ] = \underbrace{[ \begin{matrix}\underbrace{\begin{matrix}{I}_{5 \times 5} & {0}_{5 \times 5} \\ {0}_{5 \times 5} & {I}_{5 \times 5} \\ {0}_{2 \times 5} & {0}_{2 \times 5} \\ {0}_{2 \times 5} & {0}_{2 \times 5}\end{matrix}}_{{N}_{1}} & \underbrace{\begin{matrix}{K}_{1} {D}_{1} \\ {K}_{2} {D}_{2} \\ {G}_{1} {M}_{1} \\ {G}_{2} {M}_{2}\end{matrix}}_{{N}_{2}}\end{matrix} ]}_{N} [ \begin{matrix}{\tilde{\dot{x}}}_{{g}_{1} / {t}_{1}} \\ {\tilde{\dot{x}}}_{{g}_{2} / {t}_{2}} \\ {\dot{x}}_{e}\end{matrix} ] & ( 52 )\end{matrix}$$

In some embodiments, at least four modes of operation can be performed by a robotic system for surgery: intra-organ manipulation and stabilization of the organ; organ manipulation with constrained intra-organ motions (e.g., manipulation of the eye while maintaining the relative position of devices in the eye with respect to a target point inside the eye); organ manipulation with unconstrained intra-organ motion (e.g., eye manipulation regardless of the relative motions between devices in the eye and the eye); and simultaneous organ manipulation and intra-organ operation.

Further, each of the aforementioned four modes can be used to provide a dexterity evaluation. For example, intra-organ operation with organ stabilization can be used to examine the intraocular dexterity, a measure of how well this system can perform a specified surgical task inside the eye with one of its two arms. Further, for example, organ manipulation with constrained intra-organ motions can be used to evaluate orbital dexterity, a measure of how well the two arms can grossly manipulate the rotational position of eye, while respecting the kinematic constraints at the incision points and maintaining zero velocity of the grippers with respect to the retina. Still further, for example, organ manipulation with unconstrained intra-organ motion, can be used to evaluate the orbital dexterity without constraints of zero velocity of the grippers with respect to the retina. Still further, for example, simultaneous organ manipulation and intra-organ operation can be used to measure of intra-ocular and orbital dexterity while simultaneously rotating the eye and executing an intra-ocular surgical task.

It will be understood that for the analysis below both robotic arms are put to the side of the eyeball. Two incision points can be specified by angles [π/3,π/3]^{t} and [π/3,π]^{t}. The aforementioned four modes of surgical tasks can all be based on this setup.

Rewriting equation 52 using matrices M and N, equation 53 can be obtained where \dot{q}ₕ=[\dot{q}ₕ_{ᵢ}^{t},\dot{q}ₕ_{₂}^{t}]^{t} and

$${\tilde{\dot{x}}}_{g / t} = {[ {\tilde{\dot{x}}}_{{g}_{1} / {t}_{1}}^{t} , {\tilde{\dot{x}}}_{{g}_{2} / {t}_{2}}^{t} ]}^{t} .$$

Specifying \dot{x}ₑ=0 equation 53 simplifies to equation 54 and its physical meaning can be that the angular velocity of the eye is zero. Equation 54 represents the mathematical model of intra-ocular manipulation while constraining the eye.

Similarly, specifying [symbol] =0 equation 53 can simplify to equation 55. Physically this signifies that by specifying the relative velocities of the serial robot end effector with respect to the eye to be zero, equation 55 represents the mathematical model of orbital manipulation.

$$\begin{matrix}M {\dot{q}}_{h} = {N}_{1} {\tilde{\dot{x}}}_{g / t} + {N}_{2} {\dot{x}}_{e} & ( 53 ) \\ M {\dot{q}}_{h} = {N}_{1} {\tilde{\dot{x}}}_{g / t} & ( 54 ) \\ M {\dot{q}}_{h} = {N}_{2} {\dot{x}}_{e} & ( 55 )\end{matrix}$$

For intra-organ operation with organ stabilization, two modular configurations can be taken into account. In the first configuration the robotic arms can use standard ophthalmic instruments with no distal dexterity (e.g., a straight cannula capable of rotating about its own longitudinal axis). This yields a seven degree of freedom robotic arm. The Jacobian matrix for a seven degree of freedom robotic arm can be

$${J}_{{7}_{i}} = [ {B}_{i} {A}_{i} {J}_{{P}_{i}}^{- 1} , \begin{matrix}{0}_{3 \times 1} \\ {\hat{z}}_{{Q}_{i}}\end{matrix} ]$$

as in equation 56 and equation 57. In the second configuration the robotic arms employ the serial robot, therefore a kinematic model can be represented by equation 34. An intra-ocular dexterity evaluation can be used to compare the performance of the system in both these configurations (e.g., with or without the serial robot).

The method of using multiple characteristic lengths to normalize the overall Jacobian can be used for the purpose of performance evaluation. For intra-organ operation with organ stabilization, evaluating translational and rotational dexterity separately can be accomplished by investigating the upper and lower three rows of J₇_{ᵢ} and Jₕ_{ᵢ}. Equation 56 and equation 58 can give the normalized sub-Jacobians for translational motions of seven degree of freedom and eight degree of freedom robots, while equation 57 and equation 59 can give the normalized sub-Jacobians for rotational motions of seven degree of freedom and eight degree of freedom robots.

$$\begin{matrix}{J}_{7 DoF\_t} = [ {I}_{3 \times 3} , {0}_{3 \times 3} ] [ {B}_{i} {A}_{i} {J}_{{P}_{i}}^{- 1} , \begin{matrix}{0}_{3 \times 1} \\ {\hat{z}}_{{Q}_{i}}\end{matrix} ] [ \begin{matrix}{I}_{6 \times 6} & {0}_{6 \times 1} \\ {0}_{1 \times 6} & 1 / {L}_{s}\end{matrix} ] & ( 56 ) \\ {J}_{7 DoF\_r} = [ {0}_{3 \times 3} , {I}_{3 \times 3} ] [ {B}_{i} {A}_{i} {J}_{{P}_{i}}^{- 1} , \begin{matrix}{0}_{3 \times 1} \\ {\hat{z}}_{{Q}_{i}}\end{matrix} ] [ \begin{matrix}{L}_{P} {I}_{6 \times 6} & {0}_{6 \times 1} \\ {0}_{1 \times 6} & 1\end{matrix} ] & ( 57 ) \\ {J}_{8 DoF\_t} = [ {I}_{3 \times 3} , {0}_{3 \times 3} ] {J}_{{h}_{i}} [ \begin{matrix}{I}_{6 \times 6} & {0}_{6 \times 2} \\ {0}_{2 \times 6} & {I}_{2 \times 2} / {L}_{s}\end{matrix} ] & ( 58 ) \\ {J}_{8 DoF\_r} = [ {0}_{3 \times 3} , {I}_{3 \times 3} ] {J}_{{h}_{i}} [ \begin{matrix}{L}_{P} {I}_{6 \times 6} & {0}_{6 \times 2} \\ {0}_{2 \times 6} & {1}_{2 \times 2}\end{matrix} ] & ( 59 )\end{matrix}$$

Organ manipulation with constrained intra-organ motions can be used to evaluated the orbital dexterity when simultaneously using both arms to rotate the eyeball. The evaluation can be designed to address the medical professionals' need to rotate the eye under the microscope in order to obtain a view of peripheral areas of the retina.

The two arms can be predetermined to approach a target point on the retina. The relative position and orientation of the robot end effector with respect to a target point remains constant. The target point on the retina can be selected to be [5π/6, 0]^{t}, defined in the eye and attached coordinate system {E}. Frame {E} can be defined similarly as the organ coordinate system {O} and can represent the relative rotation of the eye with respect to {W}. This can cause the target point to rotate together with the eye during a manipulation.

To verify the accuracy of the derivation, a desired rotation velocity of the eye of 10°/sec about the y-axis can be specified and the input joint actuation velocities can be calculated through the inverse of the Jacobian matrix. For rotating the eye by fixing the end effector to a target point two serial robots (e.g., intra-ocular dexterity robots) and the eyeball form a rigid body allowing no relative motion in between. The rates of the serial robot joints can be expected to be zero.

For organ manipulation with unconstrained intra-organ motion, there can be no constraint applied on [symbol] . Accordingly, it can not be necessary to put limits on the velocities of point gᵢ with respect to a selected target point tᵢ. Further, inserting equation 51 into equation 53 yields:

$$\begin{matrix}M {\dot{q}}_{h} = {N}_{1} {O}_{1} {\dot{q}}_{h} + {N}_{1} {O}_{2} {\dot{x}}_{e} + {N}_{2} {\dot{x}}_{e} \\ where {O}_{1} = [ \begin{matrix}{K}_{1} {J}_{{h}_{1}} & {0}_{5 \times 8} \\ {0}_{5 \times 8} & {K}_{2} {J}_{{h}_{2}}\end{matrix} ] and {O}_{2} = [ \begin{matrix}- {K}_{1} {D}_{1} \\ - {K}_{2} {D}_{2}\end{matrix} ] . & ( 60 ) \\ ( M - {N}_{1} {O}_{1} ) {\dot{q}}_{h} = ( {N}_{1} {O}_{2} + {N}_{2} ) {\dot{x}}_{e} & ( 61 )\end{matrix}$$

For simultaneous organ manipulation and intra-organ operation, both arms coordinate to manipulate the eyeball. Further, one arm also operates inside the eye along a specified path. The overall dexterity of the robot utilizing this combined motion can be evaluated. It will be understood that assuming the eye can be rotated about the y-axis by 10°, one arm of the robotic system can scan the retina independently, meaning that there can be a specified relative motion between this arm and the eye. Assuming that the arm inserted through port [π/3, π]^{t} retains fixed in position and orientation with respect to the eye, the arm inserted through port [π/3, π/3]^{t} can coordinate with the previous arm to rotate the eye 10° about the y-axis, but it also scans the retina along the latitude circle θ=5π/6 by 60°.

Transforming the linear and angular velocities from the parallel robot platform center to frame {Qᵢ}, results in:

v_{Q}_{ᵢ}=v_{P}_{ᵢ}+ω_{P}_{ᵢ}×(\vec{pᵢqᵢ}) (62)

ω_{Q}_{ᵢ}=ω_{P}_{ᵢ} (63)

Further, writing equation 62 and equation 63 in matrix form results in the twist of the distal end qᵢ of the connection link:

\dot{x}_{Q}_{ᵢ}=Aᵢ\dot{x}_{P}_{ᵢ} (64)

where Aᵢ=W(\vec{pᵢqᵢ}) can be the twist transformation matrix.

Further, having

$${B}_{i} = W ( \vec{{q}_{i} {\dot{n}}_{i}} )$$

and Cᵢ=W(\vec{nᵢgᵢ}) the twist of point gᵢ contributed by the parallel robot platform can be calculated. By incorporating the two serial degrees of freedom of the serial robot, the twist of point gᵢ can be obtained:

$${\dot{x}}_{{G}_{i}} = {C}_{i} {B}_{i} {\dot{x}}_{{Q}_{i}} + {C}_{i} [ \begin{matrix}0 \\ {\hat{z}}_{{Q}_{i}}\end{matrix} ] {\dot{q}}_{{s}_{i} 1} + [ \begin{matrix}r {\hat{z}}_{{G}_{i}} \\ {\hat{y}}_{{N}_{i}}\end{matrix} ] {\dot{q}}_{{s}_{i} 2} \qquad ( 65 )$$

Yielding the Jacobian Jₛ_{ᵢ} of the serial robot as:

\dot{x}_{G}_{ᵢ}=CᵢBᵢ\dot{x}_{Q}_{ᵢ}+Jₛ_{ᵢ}\dot{q}ₛ_{ᵢ} (66)

where

$${J}_{{s}_{i}} = [ \begin{matrix}[ ( - \vec{{n}_{i} {g}_{i}} ) \times ] {\hat{z}}_{{Q}_{i}} & r {\hat{z}}_{{G}_{i}} \\ {\hat{z}}_{{Q}_{i}} & {\hat{y}}_{{N}_{i}}\end{matrix} ]$$

can include the speeds of rotation about the axis of the serial robot tube and the bending of the pre-curved NiTi cannula. The hybrid Jacobian matrix relating the twist of point gᵢ and all eight inputs of one arm can be obtained as in equation 34 where Jₕ_{ᵢ}=[CᵢBᵢAᵢJ_{P}_{ᵢ}⁻¹, Jₛ_{ᵢ}] and \dot{q}ₕ_{ᵢ}=[_{P}_{ᵢ}^{t}, \dot{q}ₛ_{ᵢ}^{t}]^{t}.

Further, the 5×1 Euler angle parameterization of the desired i^{th} end effector velocity, [symbol] , can be related to the general twist of the i^{th} robot end effector, [symbol] by the degenerate matrix Kᵢ. The matrix can be derived using a relationship relating the Cartesian angular velocities to the Euler angle velocities:

[ωₓ,ω_{y},ω_{z}]^{t}=Rᵢ[\dot{φ},\dot{θ},\dot{φ}]^{t} (67)

where

$${R}_{i} = [ \begin{matrix}0 & - sin ( {φ}_{i} ) & cos ( {φ}_{i} ) sin ( {θ}_{i} ) \\ 0 & cos ( {φ}_{i} ) & sin ( {φ}_{i} ) sin ( {θ}_{i} ) \\ 1 & 0 & cos ( {θ}_{i} )\end{matrix} ]$$

With the above relationship, the general twist of a system, \dot{x}, can be related to the 6×1 Euler angle twist, [\dot{x},\dot{y},ż,\dot{φ},\dot{θ},\dot{φ}]^{t}, as follows:

$${[ \dot{x} , \dot{y} , \dot{z} , \dot{φ} , \dot{θ} , \dot{φ} ]}^{t} = {S}_{i} \dot{x} \\ where {S}_{i} = [ \begin{matrix}I & 0 \\ 0 & {R}_{i}^{- 1}\end{matrix} ] . \qquad ( 68 )$$

The 5×1 Euler parameterization used in the aforementioned path planning equation can be derived by applying a 5×6 degenerate matrix to the 6×1 Euler angle twist, as follows:

[symbol]

=[I_{5×5},0_{5×1}][\dot{x},\dot{y},ż,\dot{φ},\dot{θ},\dot{φ}] (69)

Substituting the relationship between the generalized and the 6×1 Euler angle twist above yields the Matrix Kᵢ as follows:

[symbol]

=Kᵢ\dot{x} (70)

where Kᵢ=[I_{5×5},0_{5×1}]Sᵢ.

As specified above, the constraint that each insertion arm moves at the insertion point only with the velocity equal to the velocity of the organ surface at that point plus any velocity along the insertion needle can be derived as follows. To assist in the development of this constraint, point mᵢ can be defined at the insertion point on the surface of the organ and m′ᵢ can be defined as point on the insertion needle instantaneously coincident with mᵢ. The velocity of m′ᵢ must be equal to the velocity of point mᵢ in the plane perpendicular to the needle axis:

v_{m′}_{ᵢ}_{⊥}=vₘ_{ᵢ}_{⊥} (71)

Taking a dot product in the directions \hat{x}_{Q}_{ᵢ} and ŷ_{Q}_{ᵢ} yields two independent constraint equations:

\hat{x}_{Q}_{ᵢ}^{t}v_{m′}_{ᵢ}=\hat{x}_{Q}_{ᵢ}^{t}vₘ_{ᵢ} (72)

ŷ_{Q}_{ᵢ}^{t}v_{m′}_{ᵢ}=ŷ_{Q}_{ᵢ}^{t}vₘ_{ᵢ} (73)

These constraints can be expressed in terms of the joint angles and organ velocity by relating the velocities of point mᵢ and m′ᵢ to the robot and organ coordinate systems. The velocity of point m′ᵢ can be related to the velocity of frame {Qᵢ} as

v′ₘ_{ᵢ}=v_{Q}_{ᵢ}+ω_{Q}_{ᵢ}×\vec{qᵢmᵢ} (74)

By substituting the twist of frame {Qᵢ}, equation 74 becomes

v′ₘ_{ᵢ}=[I_{3×3},0_{3×3}]\dot{x}_{Q}_{ᵢ}+Eᵢ[0_{3×3},I_{3×3}]\dot{x}_{Q}_{ᵢ} (75)

where Eᵢ=[(−\vec{qᵢmᵢ})×].

Further, inserting equation 64 and equation 33 and writing in terms of the hybrid joint parameters \dot{q}ₕ_{ᵢ} yields:

v_{m′}_{ᵢ}=Fᵢ\dot{q}ₕ_{ᵢ} (76)

where Fᵢ=(I_{3×3},0_{3×3}+Eᵢ[0_{3×3},I_{3×3}])AᵢJ_{P}_{ᵢ}⁻¹[I_{6×6},0_{6×2}].

An expression for the velocity of the insertion point m; can be related to the desired organ velocity, yielding:

vₘ_{ᵢ}=\dot{x}ₒₗ+Mᵢ\dot{x}ₒₐ (77)

where Mᵢ=[(−\vec{mᵢ})×].

Further, substituting equation 76 and equation 77 into equation 72 and equation 73 yields the constraint equations given the rigid body motion of the organ-robot system:

\hat{x}_{Q}_{ᵢ}^{t}Fᵢ\dot{q}ₕ_{ᵢ}=\hat{x}_{Q}_{ᵢ}^{t}(\dot{x}ₒₗ+Mᵢ\dot{x}ₒₐ) (78)

ŷ_{Q}_{ᵢ}^{t}Fᵢ\dot{q}ₕ_{ᵢ}=ŷ_{Q}_{ᵢ}^{t}(\dot{x}ₒₗ+Mᵢ\dot{x}ₒₐ) (79)

Vectors \hat{x}_{Q}_{ᵢ} and ŷ_{Q}_{ᵢ} can be put in matrix form as Gᵢ=[\hat{Q}_{ᵢ},ŷ_{Q}_{ᵢ}]^{t}, and matrix Pᵢ can be used to denote Pᵢ=[I_{3×3},Mᵢ].

Other embodiments, extensions, and modifications of the ideas presented above are comprehended and should be within the reach of one versed in the art upon reviewing the present disclosure. Accordingly, the scope of the disclosed subject matter in its various aspects should not be limited by the examples presented above. The individual aspects of the disclosed subject matter, and the entirety of the disclosed subject matter should be regarded so as to allow for such design modifications and future developments within the scope of the present disclosure. The disclosed subject matter can be limited only by the claims that follow.

What is claimed is:

1. A tele-robotic microsurgical system for eye surgery, comprising:
a tele-robotic master and a slave hybrid-robot;
the tele-robotic master having at least two user controlled master slave interfaces;
the slave hybrid-robot having at least two robotic arms attached to a frame releasably attachable to a patient's head; and
wherein the at least two robotic arms each have a serial robot connected to a parallel robot.

2. The system of claim 1, wherein the parallel robot has six degrees of freedom and the serial robot has two degrees of freedom.

3. The system of claim 2, wherein the serial robot comprises one rotational degree of freedom about its longitudinal axis and one degree of freedom bending an end-effector.

4. The system of claim 3, wherein the end-effector comprises a cannula and a tube.

5. The system of claim 4, wherein the cannula is a NiTi cannula that bends in one degree of freedom as it is moves outside of the tube.

6. The system of claim 4, wherein the cannula is a backlash-free superelastic NiTi cannula for providing manipulation inside an eye.

7. The system of claim 5, wherein the NiTi cannula has a structural design for at least one of drug delivery, aspiration, light delivery, and delivery of at least one of micro-grippers, picks, and micro knives.

8. The system of claim 1, wherein the serial robot manipulates and stabilizes the eye while each of the robotic arms moves substantially together.

9. The system of claim 1, wherein the slave hybrid-robot has a structural configuration having at least one of tool replacement, controllable visualization inside the eye, controllable light source, drug delivery, and aspiration.

10. The system of claim 1, wherein the tele-robotic microsurgical system comprises a structure for at least one of intraocular dexeterity, dual arm dexterious manipulations inside the eye, force feedback, controllable lighting, aspiration and drug delivery, and stabilization and manipulation of the eye.

11. The system of claim 1, wherein the frame is releasably attached with at least one of a locking bite-plate and a coronal strap.

12. The system of claim 1, wherein the serial robot is releasably attached to the parallel robot.

13. The system of claim 1, wherein the at least two robotic arms are arranged to at least one of stabilizing and manipulating the eye.

14. The system of claim 1, wherein the at least two robotic arms comprise adjustable structures for adjusting into position at the initial setup of the system.

15. A tele-robotic microsurgical system for eye surgery, comprising:
a frame, a first robotic arm, a second robotic arm, and a tele-robotic master;
the frame being able to be releasably attached to an object to be operated on;
the first robotic arm and second robotic arm each comprise a parallel robot and a serial robot;
the tele-robotic master comprises a master slave user controlled interface; and
the serial robot comprises a tube and a cannula.

16. The system of claim 15, wherein at least one of the tube and cannula apply force on the eye for at least one of stabilizing, positioning, and manipulating the eye.

17. The tube of claim 16, wherein the cannula comprises a pre-bent NiTi cannula, and the cannula extending from the tube.

18. The cannula of claim 17, wherein the cannula is designed for at least one of drug delivery, aspiration, light delivery, and for delivering at least one of microgrippers, picks, and micro knives.

19. The system of claim 17, wherein at least one of the tube and the pre-bent NiTi cannula rotates about their longitudinal axis.

20. A tele-robotic microsurgical system for surgery on a hollow anatomically suspended organ, comprising:
a tele-robotic master and a slave hybrid-robot;
the tele-robotic master comprises at least one user controlled master slave interface;
the slave hybrid-robot comprises at least one robotic arm attached to a frame releasably attachable to a patient; and
the at least one robotic arm comprises a parallel robot and a serial robot.

21. The device of claim 20, wherein the parallel robot comprises a robot having six degrees of freedom and the serial robot comprises a robot having two degrees of freedom.

22. The serial robot of claim 21, further comprises a tube and a NiTi cannula that bends in one degree of freedom as it moves outside of the tube.

23. The serial robot of claim 22, wherein at least one of the tube and cannula rotate about their longitudinal axis.

24. A slave-hybrid robot for surgery on a hollow anatomically suspended organ, comprising:
a frame releasably able to be attached to a patient and at least one robotic arm is releasably attached to the frame;
the at least one robotic arm comprises a parallel robot and a serial robot;
the serial robot comprises a tube for delivering a pre-bent NiTi cannula;
at least one of the tube and the pre-bent NiTi cannula are capable of rotating about their longitudinal axis; and
the pre-bent NiTi cannula bends when extended from the tube.
